(19) United States
(12) Patent Application Publication
(10) Pub. No.: US 2013/0178750 A1
(43) Pub. Date: Jul. 11, 2013

(54) Methods and Apparatus for Regulating Blood Pressure

(71) Applicant: Volcano Corporation, San Diego, CA (US)

(72) Inventors: David M. Sheehan, Poway, CA (US); Curtis Kinghorn, Oceanside, CA (US)

(73) Assignee: Volcano Corporation, San Diego, CA (US)

(21) Appl. No.: 13/725,884

(22) Filed: Dec. 21, 2012

Related U.S. Application Data
(60) Provisional application No. 61/580,054, filed on Dec. 23, 2011.

(51) Int. Cl. A61B 5/00; A61B 5/0215

(52) U.S. Cl. CPC A61B 5/4836; A61B 5/0215
USPC 600/486; 604/9

(57) ABSTRACT

A blood pressure control apparatus, system, and methods of modifying intravascular blood flow of a patient is disclosed. In one aspect, the blood pressure control apparatus comprises an intravascular flow-modifying device including an expandable, hollow, stent-like support member configured for implantation within the vasculature, which includes an upstream sensor, a downstream sensor, and a flow restrictor. The flow restrictor is configured to partially occlude a vessel lumen and thereby artificially create back pressure upstream of the device, which causes dilation of the vessel wall and activation of the baroreceptors upstream of the device. Activation of the baroreceptors may depress the activity of the sympathetic nervous system, thereby contributing to a decrease in systemic blood pressure. The flow restrictor is also configured to partially occlude the renal vein lumen, thereby artificially increasing renal perfusion and depressing the baroreceptor-mediated sympathetic and neurohormonal efforts to raise blood pressure.

### TECHNICAL FIELD

Embodiments of the present disclosure relate generally to the field of medical devices and, more particularly, to an apparatus, systems, and methods for regulating blood pressure to affect the baroreceptor system for the treatment and/or management of various medical disorders.

### BACKGROUND

Hypertension and its associated conditions, chronic heart failure (CHF) and chronic renal failure (CRF), constitute a significant and growing global health concern. Current therapies for these conditions span the gamut covering non-pharmacological, pharmacological, surgical, and implanted device-based approaches. Despite the vast array of therapeutic options, the control of blood pressure and the efforts to prevent the progression of heart failure and chronic kidney disease remain unsatisfactory.

Hypertension, or elevated systemic blood pressure, occurs when the body's smaller blood vessels constrict, causing an increase in systemic blood pressure. Because the blood vessels constrict, the heart must work harder to pump blood through the vasculature and maintain blood flow at the higher pressures. Sustained periods of systemic hypertension may eventually result in damage to multiple organ systems, including the brain, heart, kidneys, peripheral vasculature, and others. Sustained hypertension may result in heart failure, which is characterized by an inability of the heart to pump enough blood to meet the body's requirements. Heart failure (and hypertension alone) trigger various bodily responses to compensate for the heart's inability to pump sufficient blood to the tissues. Many of these responses are mediated by an increased level of activation of the baroreceptor system, which operates without conscious control.

Blood pressure is controlled by a complex interaction of electrical, mechanical, and hormonal forces in the body that are partially orchestrated by the baroreflex system, a key mechano-electrical component of blood pressure control, as well as the sympathetic and parasympathetic nervous systems, key electrical components of blood pressure control. Throughout the body, the blood pressure is modulated at least in part by the activity of the baroreflex system, a branching network of stretch receptors extending throughout the vessel walls of the cardiovascular system. The baroreflex system connects the brain, the heart, the kidneys, and the peripheral blood vessels, each of which plays an important role in the regulation of the body's blood pressure. Baroreceptors sense stretch and pressure deformations of the vessel wall in response to changes in blood pressure. For example, an increase in blood pressure causes the arterial walls to stretch, and a decrease in blood pressure causes the arterial wall to return to original size. Baroreceptors send signals reflecting the sensed pressure conditions to the brain that cause reflexive alterations in the activity of the sympathetic and parasympathetic nervous systems, thereby contributing to adjustments in blood pressure.

The baroreflex system is one of the body's homeostatic mechanisms for maintaining blood pressure. The baroreflex system provides a negative feedback loop, in which increased blood pressure leads to increased baroreceptor activation, which ultimately leads to systemic changes throughout the body working to decrease the blood pressure. In general, increased baroreceptor activation triggers the brain to decrease the level of sympathetic nervous system (SNS) activity and increase the level of parasympathetic activity, thereby adjusting the activities of various organs to decrease the blood pressure. With increased SNS activity, the brain signals the heart to increase cardiac output, signals the kidneys to expand the blood volume by retaining sodium and water, and signals the arterioles of the peripheral vasculature to constrict to elevate the blood pressure. Thus, when baroreceptor activation inhibits SNS activity, the resulting reduction in blood volume, reduction in cardiac output, and decrease in peripheral resistance contribute to a decrease in systemic blood pressure.

FIG. 1 shows a schematic illustration of a generic arterial vessel 100 including baroreceptors 110 disposed in the vessel wall 120. A network of baroreceptors extends throughout the walls of the human vasculature, including the arterial and venous vessels. As shown in FIG. 1, the baroreceptors 100 form arbors 130 or nets extending within the vessel walls 120. In actuality, because the baroreceptors 100 may be so profusely distributed and arborized within the vessel walls 120 of the major vessels, discrete baroreceptor arbors 130 are not readily visible. To this end, those skilled in the art will recognize that the baroreceptors 110 and baroreceptor arbors 130 depicted in FIG. 1 are primarily schematic for the purposes of illustration and discussion.

The baroreceptor arbor 130 comprises a plurality of baroreceptors 110, each of which transmits signals to the brain via a nerve 140 in response to the detected stretch and/or pressure deformations of the vessel wall 120. Each baroreceptor 110 is a type of mechanical receptor, such as, by way of non-limiting example, a stretch or pressure receptor, used by the body to alert the brain to the current blood pressure at individual sites within the vasculature. The baroreceptors 100 sense pressure and/or stretch deformations of the vessel wall 120 in response to changes in local blood pressure. Typically, an increase in blood pressure causes the vessel wall 120 to stretch, and a decrease in blood pressure causes the vessel wall 120 to return to original size. Such a change in arterial wall stretch occurs with every beat of the heart, but the changes may be more pronounced and/or prolonged in conditions of sustained hypertension or hypotension. The baroreceptors 110 continuously signal the sensed local pressure condition within the vessel 100 to the brain through the nerve 140. Thus, the baroreceptors 110 send signals reflecting the sensed local pressure conditions to the brain, which causes reflexive alterations in the nervous system that modulate the systemic blood pressure.

Baroreceptors are profusely distributed in several locations throughout the arterial vasculature, including, by way of non-limiting example, the aortic arch, the carotid sinuses, the carotid arteries, the subclavian arteries, the brachiocephalic artery, and the renal arteries. Baroreceptors are also distributed throughout the venous vasculature and the cardiopulmonary vasculature, including, by way of non-limiting example, the chambers of the heart, the superior vena cava (SVC), the inferior vena cava (IVC), the jugular veins, the subclavian veins, the iliac veins, the femoral veins, and the renal veins. In addition, baroreceptors and baroreceptor-like receptors may be found in other peripheral areas such as the intrarenal juxtaglomerular apparatus of the kidney. For the purposes of this disclosure, a baroreceptor is defined as any sensor of pressure and/or stretch deformations in vessel walls secondary to changes in blood pressure or blood volume within the cardiovascular system. While there may be structural or anatomical differences among the various baroreceptors in the cardiovascular system, for the purposes of the present disclosure, activation may be directed at any of these receptors so long as they provide the desired effects of the particular application.

FIG. 2 illustrates the role of the baroreceptors 110 in the maintenance of cardiovascular homeostasis, including the control of blood pressure 145 and cardiac output 145. Changes in local blood pressure are sensed indirectly, through the baroreceptor's sensitivity to mechanical deformation during vascular stretch and/or pressurization. The resultant baroreceptor signals from the individual baroreceptors 110 are processed by the brain 150 to induce activity in a number of body systems to maintain cardiovascular homeostasis. As illustrated in FIG. 2, the baroreceptors 110, the body systems, and the requisite nervous connections therebetween may be collectively referred to as the baroreflex system 160. Throughout the body, the blood pressure is modulated at least in part by the activity of the baroreflex system 160, which is formed at least by the brain 150, the heart 165, the kidneys 170, the peripheral vessels 180, the nervous system 190, and the branching network or arbor 130 of baroreceptors 110 extending throughout the vessel walls 120 of the cardiovascular system as well as portions of the heart 165 and the kidney 170. Baroreceptors 110 send signals that reflect the sensed local pressure conditions through the nerve 140 and the nervous system 190 to the brain 150, which is therefore able to recognize changes in blood pressure, one of the indicators of cardiac output.

The baroreflex system 160 functions as a negative feedback arc wherein the level of signaling or activation of the baroreceptors 110 informs the brain about the current blood pressure conditions and the brain responds by activating or deactivating either the sympathetic or parasympathetic nervous system to preserve the cardiovascular homeostasis. Specifically, the baroreflex system 160 provides a negative feedback loop in which a sensed elevation in blood pressure reflexively causes systemic blood pressure to decrease, and a sensed decrease in blood pressure depresses the baroreflex, causing blood pressure to rise. When the blood pressure rises, the vessel wall 120 distends, resulting in stretch and pressure against the baroreceptors 110. Active baroreceptors fire action potentials or signals more frequently than inactive baroreceptors. The greater the degree of deformation or stretch, the more rapidly the baroreceptors fire action potentials.

Most baroreceptors are tonically active at mean arterial pressures (MAP) above approximately 70 mm Hg, called the baroreceptor set point. When the MAP falls below the set point, baroreceptors are essentially silent. The baroreceptor set point is not fixed; its value may change with changes in blood pressure that persist for 1-2 days. For example, in chronic hypertension, the set point may increase; on the other hand, chronic hypotension may result in a depression of the baroreceptor set point.

Stimulating the baroreceptors 110 ultimately inhibits the SNS and stimulates the parasympathetic nervous system (PNS), thereby reducing systemic arterial pressure by decreasing peripheral resistance and cardiac contractility. The sympathetic and parasympathetic branches of the autonomic nervous system have opposing effects on blood pressure. Sympathetic activation leads to increased contractility of the heart, increased heart rate, venoconstriction, increased fluid retention, and arterial vasoconstriction, all of which tend to raise blood pressure by elevating the total peripheral resistance, blood volume, and cardiac output. Conversely, parasympathetic activation leads to a decrease in heart rate and a minor decrease in contractility, resulting in decreased cardiac output and therefore a tendency to decrease blood pressure. By coupling sympathetic inhibition with parasympathetic activation, increased activation of the baroreceptors 110 may dramatically reduce blood pressure because sympathetic inhibition leads to a drop in total peripheral resistance and cardiac output, while parasympathetic activation leads to a decreased heart rate and a reduced cardiac output. Similarly, by coupling sympathetic activation with parasympathetic inhibition, the decreased activation or signaling from the baroreceptors 110 may raise blood pressure because sympathetic activation increases the total peripheral resistance, increases fluid volume, and elevates cardiac output, and parasympathetic inhibition enhances these effects.

For example, increased local blood pressure causes increased pressure or stretch of the vessel wall 120, causing increased activation or signaling of the baroreceptors 110, which leads the baroreflex system 160 to inhibit SNS activity and stimulate PNS activity to obtain an ultimate reduction in systemic blood pressure by a variety of mechanisms, such as, for example, decreasing peripheral resistance through vasodilation of the vessels 180. Conversely, when the local blood pressure is low, a decreased level of activity from the baroreceptors 110 conveys the low blood pressure to the brain 150, and the brain 150 interprets the decreased level of baroreceptor activity to mean that the cardiac output is insufficient to meet the body's demands. Consequently, the baroreflex system 160 stimulates reflexive increases in SNS activity and decreases in PNS activity that alters the behavior of various organs within the baroreflex system 160, including the heart 165, the kidneys 170, the peripheral vessels 180, thereby contributing to an increase in blood pressure to regain cardiovascular homeostasis. Specifically, the baroreflex system 160 activates the SNS and initiates a neurohormonal sequence in response to a detected drop in local blood pressure (hypotension) that signals the heart 165 to increase cardiac output by increasing the heart rate and increasing the force of contraction, signals the kidneys 170 to increase blood volume by retaining sodium and water, and signals the vessels 180 to increase blood pressure by vasoconstricting (or narrowing).

Unfortunately, the baroreflex system 160 may occasionally contribute to the exacerbation of a patient's particular cardiovascular condition or homeostatic imbalance. For example, a patient with chronic hypertension may experience local areas of paradoxically decreased blood pressure due to (1) reduced flexibility in the vessels because of atherosclerotic narrowing of the blood vessels secondary to the hypertension and (2) a reduced cardiac output because of concomitant heart failure secondary to the hypertension. In such a patient, the baroreflex system 160 may detect areas of decreased local blood pressure and activate the SNS in response to a perceived state of cardiac insufficiency that leads to an exacerbation of hypertension and possible heart failure.

Efforts to control hypertension by combating the consequences of increased SNS activity have included drug therapy and surgical intervention. Drug therapy has included the administration of medications such as centrally acting sympatholytic drugs, angiotensin converting enzyme inhibitors and receptor blockers (intended to block the renal renin-angiotensin-aldosterone system), diuretics (intended to counter the renal sympathetic mediated retention of sodium and water), and beta-blockers (intended to reduce renin release). Although the current pharmacological strategies may alleviate the symptoms of various cardiovascular and renal disorders related to sympathetic overstimulation, the strategies have significant limitations, including limited efficacy, compliance issues, and side effects. Likewise, the surgical interventions also possess various limitations. For example, surgical interventions often involve high cost, significant patient morbidity and mortality, and may not alter the natural course of the disease.

While the existing treatments may have been generally adequate for their intended purposes, they have not been entirely satisfactory in all respects. The intravascular flow-modifying devices, systems, and associated methods of the present disclosure overcome one or more of the shortcomings of the prior art.

### SUMMARY

In one aspect, the present disclosure provides a method of treating hypertension using an implanted device to regulate blood flow. In one embodiment, the method includes implanting a flow restricting device in the vasculature of a patient, sensing blood pressure, and actuating the flow restricting device in response to the sensed blood pressure to modify the flow of blood through the flow restrictor. In a further aspect, the sensor may be used to sense the blood pressure after the actuating step to determine the effect of the modification of the blood flow. In still a further aspect, the a control system can operate to control the position of the flow restricting device to maintain a relatively constant blood pressure for the patient. In yet a further aspect, the flow restricting device includes on-board sensors and a power supply and the method includes controlling the implanted device without inputs from outside the flow constricting construct. In still a further aspect, the implanted device includes a power harvesting system and the method includes harvesting power from the human body and using the harvested power to actuate the flow restricting device.

In a further embodiment, there is a provided a vascular flow regulation device. In one aspect, the flow regulation device comprises an anchoring body configured for fixed engagement with an vascular wall and a flow constriction element coupled to the anchoring body, the flow constriction element being movable between a high flow position and a low flow position. The device further includes an actuator coupled to the flow constriction element, the actuator configured to move the flow constriction element between the high flow position and the low flow position. In one aspect, the actuator may be electrically powered. In another aspect, the device may include a power supply carried by the anchoring body.

In still a further embodiment, there is provided a vascular flow regulation device having an on-board sensing system. The flow regulation device comprises an anchoring body configured for fixed engagement with an vascular wall and a flow constriction element coupled to the anchoring body, the flow constriction element movable between a high flow position and a low flow position. The flow regulation device further includes a sensing element coupled to the anchoring body and configured to detect at least one biometric parameter. In a further aspect, the sensing element generates a signal and the flow constricting device moving the flow constricting element between the high flow and low flow positions in response to the signal. In one aspect, the sensor senses blood pressure. In still a further aspect, the actuator is configured to return to the high flow condition in the absence of power.

It is to be understood that both the foregoing general description and the following detailed description are exemplary and explanatory in nature and are intended to provide an understanding of the present disclosure without limiting the scope of the present disclosure. In that regard, additional aspects, features, and advantages of the present disclosure will be apparent to one skilled in the art from the following detailed description.

### BRIEF DESCRIPTION OF THE DRAWINGS

The accompanying drawings illustrate embodiments of the devices and methods disclosed herein and together with the description, serve to explain the principles of the present disclosure.

FIG. 1 is a cross-sectional schematic illustration of baroreceptors within a vessel wall.

FIG. 2 is schematic illustration of baroreceptors within a vessel wall and a block diagram illustrating the physiologic connection between the baroreceptor system, the sympathetic nervous system, and various organ systems.

FIG. 3 is a schematic illustration of the intravascular flow-modifying device positioned in an expanded condition within a vessel lumen according to one embodiment of the present disclosure.

FIG. 4 is a schematic illustration of the intravascular flow-modifying device positioned in an expanded condition within a renal vein according to one embodiment of the present disclosure.

FIG. 5 is a schematic illustration of a blood pressure regulating system including the intravascular flow-modifying device according to one embodiment of the present disclosure positioned within the renal anatomy.

FIGS. 6a and 6b is a block diagram of the component parts of the intravascular flow-modifying device according to one embodiment of the present disclosure.

FIGS. 7a, 7b, and 7d-7f are schematic illustrations of partially cross-sectional perspective views of wirelessly communicating intravascular flow-modifying devices according to different embodiments of the present disclosure.

FIG. 7c is a schematic illustration of the intravascular flow-modifying device in an expanded condition according to one embodiment of the present disclosure.

FIG. 8a is a schematic illustration of a perspective view of the intravascular flow-modifying device in a longitudinally expanded condition according to one embodiment of the present disclosure.

FIG. 8b is a schematic illustration of a perspective view of the intravascular flow-modifying device illustrated in FIG. 8a in a longitudinally compressed condition according to one embodiment of the present disclosure.

FIG. 9 is a schematic illustration of a perspective view of the intravascular flow-modifying device positioned within a vessel according to one embodiment of the present disclosure.

FIG. 10a is a schematic illustration of a perspective view of the intravascular flow-modifying device in an expanded, activated condition according to one embodiment of the present disclosure.

FIG. 10b is a schematic illustration of a perspective view of the intravascular flow-modifying device shown in FIG. 10a in an expanded, partially activated condition according to one embodiment of the present disclosure.

FIG. 10c is a schematic illustration of a perspective view of the intravascular flow-modifying device shown in FIG. 10a in an expanded, unactivated condition according to one embodiment of the present disclosure.

FIG. 10d is an illustration of a plan view of the disc of the intravascular flow-modifying device shown in FIG. 10a according to one embodiment of the present disclosure.

FIG. 11a is a schematic illustration of a partially cross-sectional perspective view of a portion of the intravascular flow-modifying device shown in FIG. 10a according to one embodiment of the present disclosure.

FIG. 11b is a schematic illustration of a tab positioned in a recess in a reduced flow position of the intravascular flow-modifying device shown in FIG. 10a according to one embodiment of the present disclosure.

FIG. 11c is a schematic illustration of a tab positioned in a recess in an increased or normal flow position of the intravascular flow-modifying device shown in FIG. 10a according to one embodiment of the present disclosure.

FIG. 12a is a schematic illustration of a perspective view of the intravascular flow-modifying device in an expanded, activated condition according to one embodiment of the present disclosure.

FIG. 12b is a schematic illustration of a perspective view of the intravascular flow-modifying device shown in FIG. 12a in an expanded, partially activated condition according to one embodiment of the present disclosure.

FIG. 12c is a schematic illustration of a perspective view of the intravascular flow-modifying device shown in FIG. 12a in an expanded, unactivated condition according to one embodiment of the present disclosure.

FIGS. 13a-c are schematic illustrations of perspective views of the intravascular flow-modifying device in an expanded, unactivated condition according to one embodiment of the present disclosure.

FIG. 13d is a schematic illustration of a perspective view of the intravascular flow-modifying device shown in FIG. 13a in an expanded, activated condition according to one embodiment of the present disclosure.

FIG. 14a is a schematic illustration of a perspective view of the intravascular flow-modifying device in an expanded, activated condition according to one embodiment of the present disclosure.

FIG. 14b is a schematic illustration of a perspective view of the intravascular flow-modifying device shown in FIG. 14a in an expanded, partially activated condition according to one embodiment of the present disclosure.

FIG. 15a is a schematic illustration of a perspective view of the intravascular flow-modifying device in an expanded, unactivated condition according to one embodiment of the present disclosure.

FIG. 15b is a schematic illustration of a perspective view of the intravascular flow-modifying device shown in FIG. 15a in an expanded, activated condition according to one embodiment of the present disclosure.

FIG. 16 provides a schematic flowchart illustrating methods of positioning and controlling blood pressure and the baroreceptor system using the blood pressure control system and the intravascular flow-modifying device.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of the disclosure is intended. Any alterations and further modifications to the described devices, instruments, methods, and any further application of the principles of the present disclosure are fully contemplated as would normally occur to one skilled in the art to which the disclosure relates. In particular, it is fully contemplated that the features, components, and/or steps described with respect to one embodiment may be combined with the features, components, and/or steps described with respect to other embodiments of the present disclosure. For simplicity, in some instances the same reference numbers are used throughout the drawings to refer to the same or like parts.

The present disclosure relates generally to apparatuses, systems, and methods using intravascular flow-modifying devices for the treatment of various cardiovascular diseases, including, by way of non-limiting example, hypertension, chronic heart failure, and/or chronic renal failure. In some instances, embodiments of the present disclosure are configured to manipulate the baroreceptor system, including, by way of non-limiting example, the renal baroreceptor system, to increase or decrease sympathetic activity. In particular, renal baroreceptor activation of the sympathetic nervous system may worsen symptoms of hypertension, heart failure, and/or chronic renal failure by causing increased renal vascular resistance, renin release, and fluid retention, all of which exacerbate hypertension.

Modulation of the renal baroreceptor system using an intravascular flow-modifying device may affect renal sympathetic activity by creating localized increases and drops in blood pressure to activate and/or inactivate the baroreceptors that encircle the renal vessels, including both the arteries and the veins, as well as the intrarenal baroreceptors. By using an intravascular flow-modifying device to selectively manipulate renal baroreceptor activity, a user may affect the activity of the sympathetic nervous system (SNS) and thereby affect the activities of various organs, including the brain, heart, kidneys, and peripheral vasculature, to ultimately control the patient's systemic blood pressure.

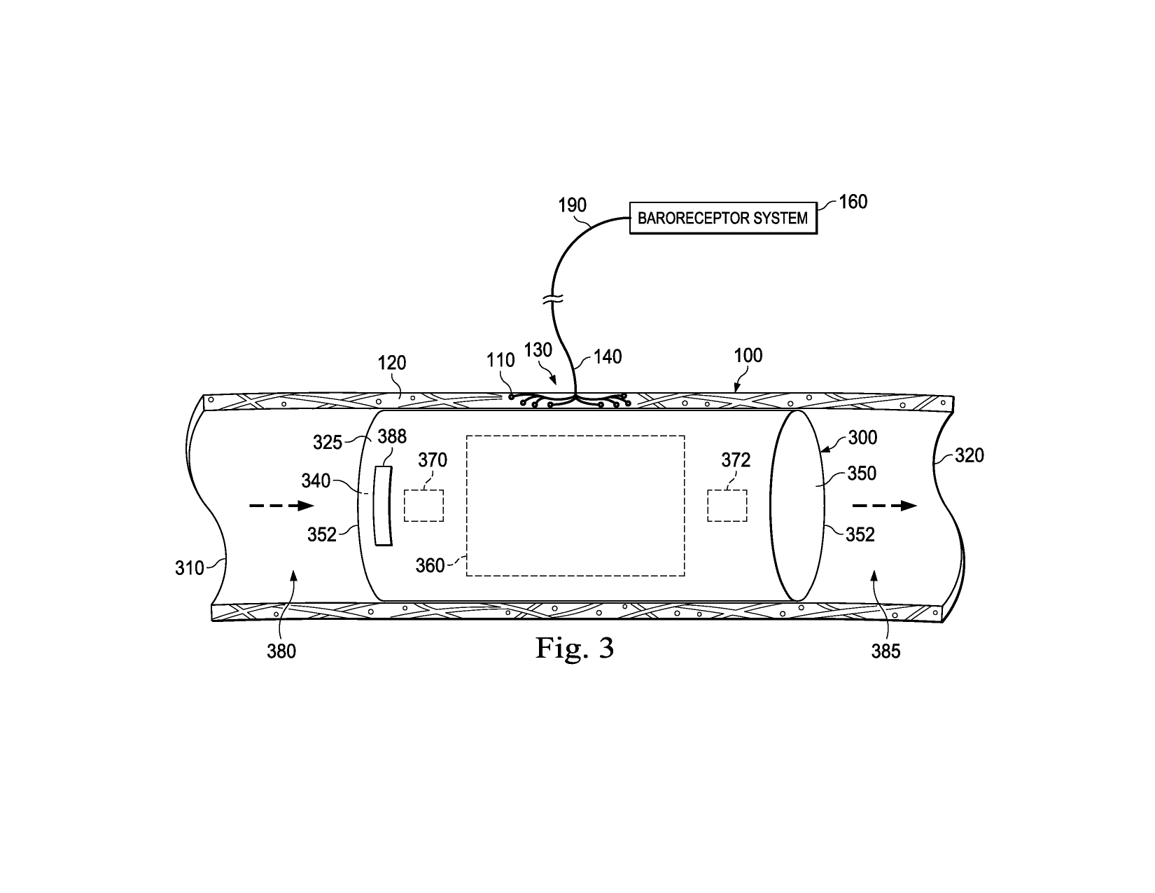

FIG. 3 illustrates an intravascular flow-modifying device 300, which is configured to affect local blood pressure by restricting blood flow and creating focal areas of increased back pressure, in an expanded condition and implanted within the generic vessel 100. The flow-modifying device 300 is shown positioned within the vessel 100 adjacent to the vessel wall 120, which contains the arbor 130 of baroreceptors 110 connected to the remainder of the baroreceptor system 160 through the nerve 140 and the nervous system 190. Blood flows through the vessel 100 from a upstream portion 310 to a downstream portion 320, as indicated by the dashed arrow. The device 300, including a support member 325 having an upstream end 340 and a downstream end 350, is positioned within a lumen 330 of the vessel 100 immediately distal to the baroreceptors 110. In alternative embodiments, the flow-modifying device 300 may be positioned anywhere within the vicinity of baroreceptors and baroreceptor-like receptors. Preferably, the ends 340, 350 have sloped or curved circumferential edges 352 to facilitate the movement of blood through the recess and prevent stagnation of blood flow within the recess.

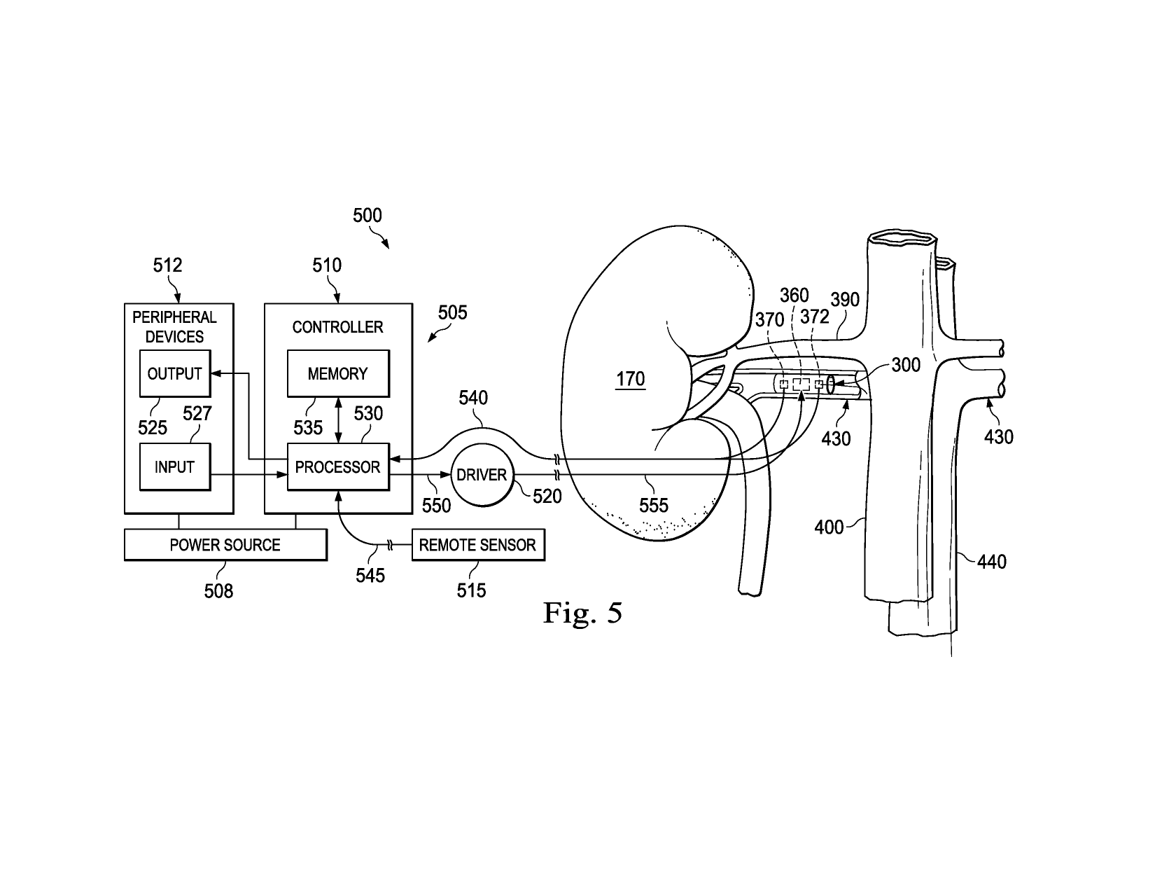

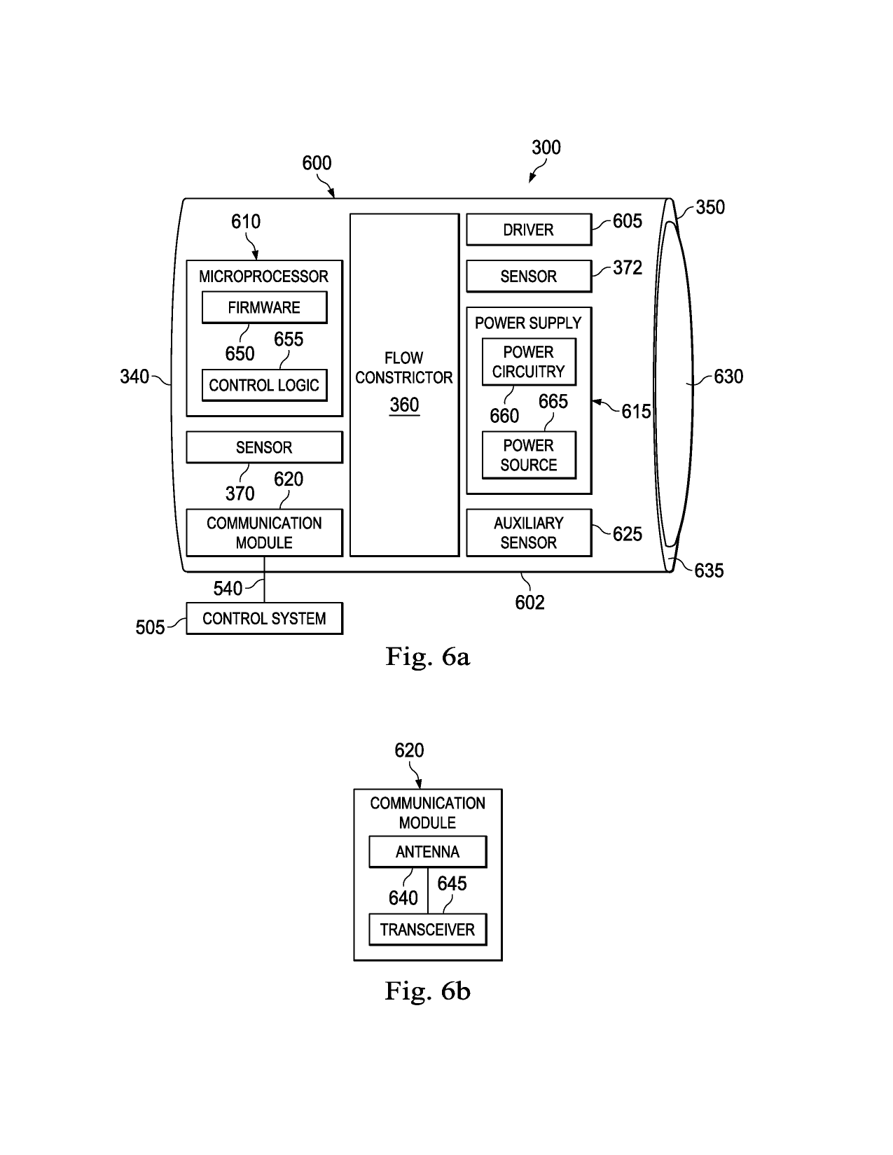

The device 300 includes a flow restrictor 360, which is configured to regulate blood flow through the device 300, at least one upstream sensor 370, and at least one downstream sensor 372, and a support member 375. The sensors 370, 372 are configured to sense and/or monitor one or more properties of blood flow, pressure, or function. As used herein, perfusion, blood perfusion, and renal perfusion generally refer to a fluid dynamic property of blood flow such as volumetric flow rate, flow velocity, and/or pressure, including absolute, mean or pulse pressure, or a fluid static property such as interstitial pressure. The upstream sensor 370 and the downstream sensor 372 are discussed in more detail below with respect to FIGS. 5 and 6a.

A user may activate or deactivate the intravascular flow-modifying device 300 to affect the local blood pressure in an upstream area 380 immediately proximal to the device 300 and thereby modulate the activation of the baroreceptors 110 located adjacent to the upstream area 380. Modulation of the baroreflex system 160 by using the intravascular flow-modifying device 300 to regulate the local blood pressure in the upstream area 380 has the potential to impact cardiovascular homeostasis by affecting the activities of individual organ systems within the baroreflex system 160, including, for example, the mechanical and hormonal activities of the heart, the kidneys, and the vessels. When the flow restrictor 360 is activated in response to a user command, a control system command, and/or sensed data from at least the sensors 370 and/or 372, the device 300 functions to partially restrict or occlude blood flow through the device from the proximal end 340 to the distal end 350. By at least partially occluding the vessel lumen distal (or downstream) of the baroreceptors 110, the back pressure is created proximal (or upstream) of the device 300 such that the vessel wall 120 expands to activate the baroreceptors 110.

For example, a user may create a local increase in blood pressure in the upstream area 380, the vicinity of the baroreceptors 110, by activating the flow restrictor 360 to partially occlude blood flow, which creates back pressure at the upstream area 380 to mechanically activate the baroreceptors 110 by stretching or otherwise deforming them as the vessel wall 120 dilates proximal to the intravascular flow-modifying device 300 to accommodate the back pressure and increased blood perfusion in the area 380.

In some embodiments, the upstream sensor 370 detects blood perfusion characteristics of the vessel 100 at the upstream area 380, and the downstream sensor detects blood perfusion characteristics of the vessel 100 at the downstream area 385. In some embodiments, the flow restrictor 360 may be activated or deactivated by the user or a processor in response to any of the sensed blood perfusion characteristics of the upstream sensor 370 and/or the downstream sensor 372. In some embodiments, the flow restrictor 360 may be slaved to the upstream sensor 370 and/or the downstream sensor 372 such that the flow resistor is activated or deactivated in response to any of the sensed blood perfusion parameters or other sensed characteristics of the upstream sensor 370 and/or the downstream sensor 372.

In some embodiments, the intravascular flow-modifying device 300 includes at least one radiopaque marker 388 to aid in positioning the device 300 in the vasculature of the patient. In some embodiments, the radiopaque marker 388 may be spaced along device 300 at a specific and known distance from the ends 340, 350. The radiopaque marker 388 may aid the user in visualizing the path and ultimate positioning of the device 300 within the vasculature of the patient. In addition, the radiopaque marker 388 may provide a fixed reference point for co-registration of various imaging modalities and treatments, including by way of non-limiting example, external imaging and/or imaging by an internal imaging apparatus (e.g., IVUS). In alternate embodiments, the some or all of component parts of the device 300 are radiopaque to aid in positioning the device 300 in the vasculature of the patient. Other embodiments may lack radiopaque markers.

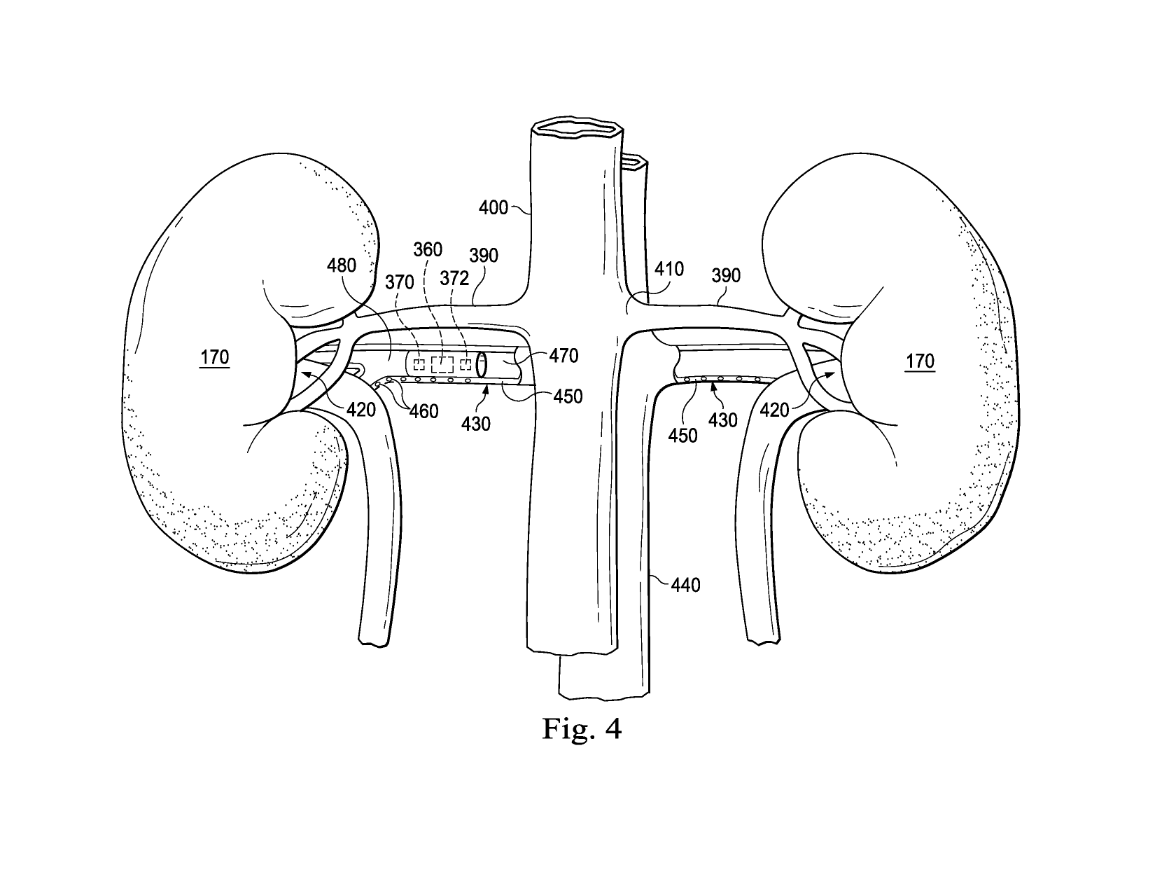

FIG. 4 shows a schematic illustration of the intravascular flow-modifying device 300 positioned within the renal anatomy. The human renal anatomy includes kidneys 170 that are supplied with oxygenated blood by right and left renal arteries 390, each of which branch off an abdominal aorta 400 at the renal ostia 410 to enter a hilum 420 of each kidney 170. The abdominal aorta 400 connects the renal arteries 390 to the heart (not shown). Deoxygenated blood flows from the kidneys 170 to the heart via right and left renal veins 430 and an inferior vena cava 440.

Specifically, the intravascular flow-modifying device 300 is shown positioned in the right renal vein 430 adjacent to the venous wall 450. Baroreceptors 460 include the baroreceptors located within a portion of the venous wall 450 located near the right hilum 420 and/or the baroreceptor-like receptors located within the juxtaglomerular apparatuses of the intrarenal vasculature. Other baroreceptors or baroreceptor-like receptors may be located in the vessel walls of the renal arteries 390, the abdominal aorta 400, the left renal vein 430, and in the juxtaglomerular apparatuses found in intimate association with the intrarenal vasculature (not shown). The device 300 is positioned within a lumen 470 of the right renal vein 430 at a location distal to the baroreceptors 460. In alternative embodiments, the flow-modifying device 300 may be positioned anywhere within the vicinity of baroreceptors, including, but not by way of limitation, the renal arteries 390, the left renal vein 430, the aorta 400, the aortic arch (not shown), the carotid arteries (not shown), and/or the IVC 440, provided the flow regulation produces the desired cardiovascular effect.

In the case of chronic hypertension and/or heart failure, the kidneys 170 may interpret decreased blood perfusion in the renal arteries 390, renal veins 430, and other parts of the intrarenal vasculature as reflecting the heart's inability to pump sufficient blood. Renal baroreceptors 460 respond to this to condition by activating and/or contributing to a SNS-mediated neurohormonal sequence that signals the heart to increase the heart rate and the force of contraction to increase the cardiac output, signals the kidneys 170 to expand the blood volume by retaining sodium and water, and signals the arterioles to constrict to elevate the blood pressure. Further, an increase in renal sympathetic activity leads to the increased renal secretion of renin, which activates a cascade of events, including vasoconstriction, elevated heart rate, and fluid retention, through the renin-angiotensin-aldosterone system (RAAS). Vasoconstriction of the renal vasculature causes decreased renal blood flow, which prompts the kidneys 170 to send afferent SNS signals to the brain, triggering peripheral vasoconstriction and exacerbating hypertension. The kidney 170 also produces cytokines and other neurohormones in response to elevated sympathetic activation that can be toxic to other tissues, particularly the blood vessels, heart, and kidney.

Thus, the cardiac, renal, and vascular responses to increased SNS activity triggered by low renal perfusion cooperate to increase the workload of the heart, creating a vicious cycle of cardiovascular injury that accelerates cardiovascular damage and exacerbates heart failure. The present disclosure addresses this kidney-mediated propagation of hypertension by providing a number of intravascular flow-modifying devices by which the kidneys may experience normal or supranormal perfusion even in the face of hypertension (and consequent reduced cardiac output and/or vasoconstriction). By maintaining or augmenting renal perfusion using a flow-modifying device, the renal baroreceptors and the baroreceptor-like receptors of the juxtaglomerular apparatus proximal of the flow-modifying device may be modulated to prompt a decrease in blood pressure, and the viscous cycle referred to above may be stopped or at least moderated to facilitate a return to normal blood pressure.

By activating the flow restrictor 360 of the intravascular flow-modifying device 300 to partially occlude the outflow of blood from the right kidney 170, a user may create an area of artificially increased blood pressure and perfusion in the intrarenal vasculature of the kidney 170 and an area 480 of the right renal vein 430 proximal to the device 300. Renal perfusion and pressure may be artificially increased, thereby increasing the activation of the baroreceptors 460 and reducing activation of the SNS to ultimately reduce systemic blood pressure. In addition, by increasing renal perfusion, the device 300 may function to increase interstitial pressure to reduce sodium and water absorption, thereby decreasing blood volume and contributing to a decrease in systemic blood pressure.

FIG. 5 illustrates a blood pressure control system 500 according to one embodiment of the present disclosure that is configured to selectively restrict intravascular blood flow to regulate local blood pressures in order to modulate the activity of the baroreflex system and contribute to the maintenance of cardiovascular homeostasis. With respect to the embodiment pictured in FIG. 5, the system 500 comprises the intravascular flow-modifying device 300, a control system 505, which includes a controller 510 and peripheral devices 512, at least one optional remote sensor 515, and a driver 520.

In FIG. 5, for the purposes of illustration only, the device 300, which is configured for intravascular placement and/or implantation and includes the upstream sensor 370 and the downstream sensor 372, is shown positioned within the right renal vein 430. Therefore, blood will flow from the right kidney 170 through the device 300 and into the IVC 440. The sensors 370, 372 are positioned on or in intimate association with the device 300. In the pictured embodiment, the sensors 370, 372 are positioned on the device 300 such that the sensor 370 may measure cardiovascular characteristics within an upstream area of the device 300 and the sensor 372 may measure cardiovascular characteristics within a downstream area of the device 300.

In alternate embodiments, the device 300 may be positioned at any intravascular location and/or site within the cardiovascular system located in the vicinity of baroreceptors. Examples of suitable arterial wall locations include, without limitation, a carotid arterial wall, an aortic arterial wall, a subclavian arterial wall, a brachiocephalic arterial wall, a renal arterial wall, a hepatic arterial wall, a splenic arterial wall, a pancreatic arterial wall, a jugular arterial wall, a femoral arterial wall, an iliac arterial wall, a pulmonary arterial wall, a brachial arterial wall, a cardiac arterial wall, a popliteal arterial wall, a tibial arterial wall, a celiac arterial wall, an axillary arterial wall, a radial arterial wall, an ulnar arterial wall, and a mesenteric arterial wall. Examples of suitable venous wall locations include, without limitation, a hepatic venous wall, an inferior vena cava venous wall, a superior vena cava venous wall, a jugular venous wall, a subclavian venous wall, an iliac venous wall, a femoral venous wall, a pulmonary venous wall, a splenic venous wall, a renal venous wall, a pancreatic venous wall, a cephalic venous wall, a tibial venous wall, an axillary venous wall, a brachial venous wall, a popliteal venous wall, a cardiac venous wall, and a brachiocephalic venous wall.

The exemplary control system 505 generally operates in the following manner. The upstream sensor 370, the downstream sensor 372, and/or the remote sensor 515 sense and/or monitor a parameter (e.g., a cardiovascular characteristic, component, or flow measurement) indicative of the need to modify the baroreflex system and generate a signal indicative of the parameter. In some instances, the user may input command signals into the control system 505. The control system 505 generates a control signal as a function of the received sensor and/or command signals. The control signal activates, deactivates, or otherwise modulates the intravascular flow-modifying device 300. Typically, activation of the device 300 results in activation of the baroreceptors 110 within the adjacent vessel wall 120 (as shown in FIG. 3). In the pictured embodiment, activation of the device 300 results in activation of the baroreceptors 460 within the renal vein wall 450 (as shown in detail in FIG. 4). In alternate embodiments, deactivation or modulation of the device 300 may cause or modify activation of the baroreceptors 110.

The intravascular flow-modifying device 300 may comprise a wide variety of devices which utilize mechanical, electrical, thermal, chemical, biological, hormonal, or other means to activate and/or deactivate the baroreceptors 110. The device 300, as mentioned above with respect to FIGS. 3 and 4, includes the upstream sensor 370, the downstream sensor 372, and the flow restrictor 360. When the sensors 370, 372, and/or 515 detect a parameter indicative of the need to modify the baroreflex system activity (e.g., excessive blood pressure), the control system 505 will typically generate a control signal to activate the device 300, thereby inducing a baroreceptor 110 signal that is perceived by the brain to be a particular blood pressure state (e.g., hypertension). When the sensors 370, 372, and/or 515 detect a parameter indicative of normal cardiovascular activity (e.g., normal blood pressure), the control system 505 may generate a control signal to partially or completely deactivate the intravascular flow-modifying device 300.

The sensors 370, 372, and 515 may comprise any suitable sensing device that measures, senses, and/or monitors a cardiovascular parameter indicative of the need to modify the activity of the baroreceptor system by modulating the activity of the device 300. For example, the sensors 370, 372, and 515 may comprise a physiologic measurement device that measures blood pressure (systolic, diastolic, average, and/or pulse pressure), blood volumetric flow rate, blood flow velocity, blood pH, gas or element content (such as, by way of non-limiting example, oxygen, carbon dioxide, and/or nitrogen content, mixed venous oxygen saturation), ECG, respiratory rate and/or respiratory efficiency, hemodynamic factors (such as, by way of non-limiting example, hormones and/or enzymes (e.g., renin, angiotensin, angiotensinogen), blood glucose, inflammatory mediators, cardiac enzymes, and/or tissue factors), vasoactivity, nerve activity, and/or tissue activity or composition. Exemplary sensors 370, 372, and 515 include, without limitation, ultrasonic sensors, flow sensors, pressure sensors, thermal sensors, blood temperature sensors, electrical contact sensors, conductivity sensors, electromagnetic detectors, chemical or hormonal sensors, pH sensors, and infrared sensors. Specific examples of suitable measurement devices for the sensors 370, 372, and 515 include a piezoelectric pressure transducer, an ultrasonic flow velocity transducer, an ultrasonic volumetric flow rate transducer, a thermodilution flow velocity transducer, a capacitive pressure transducer, a membrane pH electrode, an optical detector, and/or a strain gauge. Examples of additional suitable measurement devices for the remote sensor 515 include external devices such as, by way of non-limiting example, ECG electrodes and a blood pressure cuff. The sensors 370, 372 are described in more detail below with respect to FIG. 6.

Numerous commercially available and experimental sensor devices are suitable for use in the embodiments of the present disclosure. By way of illustration only and without limitation to the incorporation of alternative physiologic sensing devices, a selection of such physiologic sensors can be found in U.S. Pat. Nos. 5,535,752; 5,967,986; 6,152,885; 6,113,553; 6,277,078; 6,383,144; 6,430,440 and 6,411,849, each of which is hereby incorporated by reference in its entirety. In addition to electrically based sensors to detect blood flow, pressure, temperature and turbulence, suitable implantable physicologic sensors may include either alone or in combination with electrically based sensors set forth above, chemical sensors or biologic sensors to monitor constituent levels of metabolites, analytes, electrolytes, and/or proteins in the blood. By way of illustration only and without limitation to the incorporation of alternative physiologic sensing devices, a selection of such chemical and biologic sensors can be found in U.S. Pat. Nos. 6,122,536; 5,833,603; 6,673,596; 6,625,479 and 6,201,980, each of which is hereby incorporated by reference in its entirety.

In addition, the sensors and other components of the embodiments described herein may include anti-scarring agents to inhibit scarring that may occur when implanted in the body. U.S. Pub. No. 2010/0092536 entitled “Implantable Sensors and Implantable Pumps and Anti-Scarring Agents” discloses a number of suitable compounds and is hereby incorporated by reference in its entirety.

The remote sensor 515 may be positioned separate from the device 300 or combined therewith. The sensor 515 may be disposed inside the patient's body or outside the body, depending on the type of measurement device used. For example, the remote sensor 515 may be positioned in or on a blood vessel and/or organ, such as, by way of non-limiting example, a chamber of the heart, an artery such as the aortic arch, the abdominal aorta 400, a common carotid artery, a subclavian artery, or the brachiocephalic artery, or a vein such as the IVC 440, such that at least one cardiovascular parameter of interest may be readily sensed. In alternate embodiments, the sensor may be disposed, by way of non-limiting example, around an arm of the patient, against the skin of a patient, or around the finger of a patient. In some embodiments, multiple remote sensors of the same or different types may be positioned at the same or various sites in and/or on the body of the patient to obtain several measurements of one or more cardiovascular parameters from various locations within/on the patient's body.

In the pictured embodiment in FIG. 5, the control system 505 includes a power source 508, the controller 510, and the peripheral devices 512. The power source 508 may be a rechargeable battery, such as a lithium ion or lithium polymer battery, although other types of batteries may be employed. In other embodiments, any other type of power cell is appropriate for power source 508. The power source 508 provides power to the system 500, and more particularly to the control system 505 and/or the driver 520. The power source 508 may be an external supply of energy received through an electrical outlet. In some examples, sufficient power is provided through on-board batteries and/or wireless powering. In some embodiments, the power source 508 provides power to the control system 505 as well as the driver 520 and/or the device 300. In other embodiments, the power source 508 provides power to only the control system 505.

The controller 510 may be in communication with and may perform specific user-directed control functions targeted to a specific device or component of the system 500, such as the driver 520, the sensors 370, 372, and/or 515, the flow restrictor 360, and/or the intravascular flow-modifying device 300. In the pictured embodiment, the peripheral devices 512 comprise an output device 525 and an input device 527, and the controller 510 comprises a processor 530 and a memory 535.

The various peripheral devices 512, including the output device 525 and the input device 527, may enable or improve input/output functionality of the processor 530. The input device 527 includes, but is not necessarily limited to, standard input devices such as a mouse, joystick, keyboard, etc. A user may enter information into the input device 527 about the patient, such as age, weight, height, diagnosis, medications, treatments, and so forth. The processor 530 may then determine the proper therapeutic thresholds using the user input data and algorithms stored in the processor 530 and/or the memory 535. The patient-specific thresholds may be stored on the memory 535 for comparison to sensed or measured physiological characteristics.

The output device 525 includes, but is not necessarily limited to, standard output devices such as a printer, speakers, a projector, graphical display screens, etc. The output device 525 may be configured to display sensed physiological data about the patient, operational/status/mode information about the system 500, and/or alarm indications. For example, the output device 525 may include a display, a haptic surface, and/or a speaker to provide a visual, a tactile, and/or an audible alarm, respectively, in the event that the patient's sensed physiological parameters are not within a normal range, as defined based on the particular patient's medical history and condition as well as on general population guidelines. Such ranges may be calculated or created to define any of a variety of ranges, including therapeutic range (e.g., to modulate the baroreceptor system) and/or a safety range (e.g., to maintain perfusion to tissues downstream of the device 300).

The peripheral devices 525 may also comprise a CD-ROM drive, a flash drive, a network connection, and electrical connections between the processor 530 and various components of the system 500. By way of non-limiting example, the processor 530 may manipulate signals from the input 527 and/or the sensors 370, 372 to generate an graphical representation of input data (entered and sensed) on a display screen-type output device 525, may coordinate subsequent activation/deactivation of the device 300, and may store the data and the subsequent treatment plan in the memory 535. The peripheral devices 512 may also be used for downloading software containing processor instructions to enable general operation of the device 300, and for downloading software implemented programs to perform operations to control, for example, the operation of any auxiliary devices associated with and/or attached to the device 300 (e.g., the remote sensor 515).

The processor 530 is typically an integrated circuit with power, input, and output pins capable of performing logic functions. The processor 530 may include any one or more of a microprocessor, a controller, a digital signal processor (DSP), an application specific integrated circuit (ASIC), a field-programmable gate array (FPGA), or equivalent discrete or integrated logic circuitry. In some examples, processor 530 may include multiple components, such as any combination of one or more microprocessors, one or more controllers, one or more DSPs, one or more ASICs, or one or more FPGAs, as well as other discrete or integrated logic circuitry. The functions attributed to processor 530 herein may be embodied as software, firmware, hardware or any combination thereof.

The processor 530 may include one or more programmable processor units running programmable code instructions for implementing the thermal neuromodulation methods described herein, among other functions. The processor 530 may be integrated within a computer and/or other types of processor-based devices suitable for a variety of intravascular applications, including, by way of non-limiting example, baroreceptor stimulation, flow regulation, and intravascular imaging. The processor 530 may receive input data from the input device 527, from the device 300, and/or from the at least one remote sensor 515 via physical connections or wireless mechanisms. The processor 530 may use such input data to generate control signals to control or direct the operation of the driver 520 and/or the device 300. In some embodiments, the user can program or direct the operation of the device 300, the driver 520, and/or the remote sensor 515 from the controller 510 and/or the input device 527. In some embodiments, the processor 530 is in direct wireless communication with the device 300, the driver 520, and/or the remote sensor 515, and can receive data from and send commands to the device 300, the driver 520, and/or the remote sensor 515.

In various embodiments, the processor 530 is a targeted device controller that may be connected to the power source 508, the peripheral devices 512, the memory 335, the driver 520, the remote sensor 515, and/or the intravascular flow-modifying device 300. In such a case, the processor 530 is in communication with and performs specific control functions targeted to a specific device or component of the system 500, such as the device 300, without utilizing user input from the input device 527. For example, the processor 530 may direct or program the device 300 to function for a period of time in a certain pattern of activation/deactivation without specific user input to the controller 510. In some embodiments, the processor 530 is programmable so that it can function to simultaneously control and communicate with more than one component of the system 500, including the peripheral devices 512, the power source 508, the driver 520, the memory 535, and/or the device 300. In other embodiments, the system includes more than one processor and each processor is a special purpose controller configured to control individual components of the system. In some embodiments, the processor may include a plurality of processing units employed in a wide range of centralized or remotely distributed data processing schemes.

The memory 535 is typically a semiconductor memory such as, by way of non-limiting example, read-only memory, a random access memory, and/or other computer storage media. The memory 535 interfaces with processor 530 such that the processor 530 can write to and read from the memory 535. For example, the processor 530 can be configured to read data from the device 300 and/or the remote sensor 515 and write that data to the memory 535. In this manner, a series of data readings can be stored in the memory 535. The memory 535 may contain data related to the sensor signals from sensors 370, 372, and/or 515, the command signals generated by the processor 530, and/or the values and commands provided by the input device 527. The processor 530 may be capable of performing basic memory management functions, such as erasing or overwriting the memory 535, detecting when the memory 535 is full, and other common functions associated with managing semiconductor memory.

Any computer-readable media may be used in the system as the memory 535 for data storage. Computer-readable media are capable of storing information that can be interpreted by the processor 530. This information may be data or may take the form of computer-executable instructions, such as software applications, that cause the processor 530 to perform certain functions and/or computer-implemented methods. Depending on the embodiment, such computer-readable media may comprise computer storage media and communication media. Computer storage media includes volatile and non-volatile, removable and non-removable media implemented in any method or technology for storage of information such as computer-readable instructions, data structures, program modules, or other data. Computer storage media includes, but is not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by components of the system 500.

The processor 530 and/or the memory 535 may also include software containing one or more algorithms defining one or more functions or relationships between the command signals and the sensor signals. The algorithm may dictate activation or deactivation command protocols/signals depending on the received sensor signals or mathematical derivatives thereof. The algorithm may dictate an activation or deactivation control signal when a particular sensor signal falls below a predetermined threshold value, rises above a predetermined threshold value, or when the sensor signal indicates a specific physiologic event or condition.

As mentioned above, the intravascular flow-modifying device 300 may be configured to activate baroreceptors mechanically, electrically, thermally, chemically, biologically, or otherwise. In some embodiments, the blood pressure control system 500 includes the driver 520 to provide the appropriate power mode for the device 300. For example, if the device 300 utilizes pneumatic or hydraulic actuation, the driver 520 may comprise a pressure/vacuum source and the driver cable 555 may comprise a fluid/gas line(s). In the alternative, if the device 300 utilizes electrical or thermal actuation, the driver 520 may comprise a power amplifier or the like and the driver cable 555 may comprise an electrical lead(s). In the alternative, if the device 300 utilizes chemical or biological actuation, the driver 520 may comprise a fluid/chemical reservoir and a pressure/vacuum source, and the driver cable 555 may comprise a fluid/gas line(s). In the alternative, if the device 300 utilizes imaging or ultrasonic actuation, the driver 520 may comprise an ultrasound energy generator.

Under the user-directed or automated (algorithm-based) operation of the controller 510, the driver 520 may generate a selected form and magnitude of energy (e.g., a particular energy frequency) best suited to a particular application. The user may use the input device 527 and the controller 510 to initiate, terminate, and adjust various operational characteristics of the driver 520. Under the control of the user or an automated control algorithm in the processor 530, the driver 520 generates a desired form and magnitude of energy. The driver 520 may be utilized with any of the intravascular flow-modifying devices described herein for delivery of energy with the desired field parameters, i.e., parameters sufficient to induce activation and/or deactivation of the device to modify intravascular flow and thereby modulate the baroreceptor system. It should be understood that the intravascular flow-modifying devices described herein may be connected, electrically or otherwise, to the driver 520 even through the driver 520 is not explicitly shown or described with respect to each embodiment.

In the pictured embodiment, the driver 520 is located external to the patient. In other embodiments, the driver 520 may be positioned internal to the patient. In some embodiments utilizing an intravascular flow-modifying device, for example, the driver 520 may be a component part of the device 300 itself, as discussed below with respect to FIG. 6. In other embodiments, the driver 520 may not be necessary, particularly if the processor 530 and/or the device 300 itself generates a sufficiently strong electrical signal for low level electrical or thermal actuation of the device 300. In some embodiments, for example, the driver may additionally comprise or may be substituted with an alternative energy generator, such as, by way of non-limiting example, a thermoelectric polymer or a dielectric elastomer structure configured to produce energy. The control and direction of the energy supplied by the driver 520 will be described in further detail below with respect to FIG. 6a.

In various embodiments, the controller 505 may be operatively coupled to the flow-modifying device 300 by way of electric control cables or leads, wireless communication mechanisms, or a combination thereof. In addition, the controller 505 may be implanted in whole or in part within the body of the patient. In some embodiments, the entire controller 505 may be carried externally with the patient either (1) utilizing wireless communication between the device 300 and the controller 505, or (2) utilizing transdermal connections between the device 300 and the controller 505. For example, the controller 505 and/or the driver 520 may comprise an external control device or handheld programming device to operate and/or power the intravascular device 300. Alternatively, the controller 505 and the driver 520 may be implanted in the body of the patient (e.g., subcutaneous implantation) while the peripheral devices 512, which may be coupled to the controller 505 via transdermal connections, may be carried externally. As a further alternative, the transdermal connections may be replaced by wireless communication methods, such as, by way of nonlimiting example, cooperating transmitters and receivers positioned on various components of the system 500 to allow remote communication between various components of the system 500. Such wireless communication methods will be described in more detail below in relation to FIGS. 6a and 6b.

In some embodiments, the system 500 may be configured to include a plurality of electrical connections, each electrically coupled to a different component (e.g., an electrode, a sensor, and/or a flow restrictor) on the device 300 via a dedicated conductor and/or a sensor cable, running transdermally and/or intravascularly between the device 300 to the control system 505 and/or the driver 520. Such a configuration may allow for a specific group or subset of components on the device 300 to be easily energized or powered by the driver 520. Such a configuration may also allow the device 300 to transmit data from any of a variety of sensors to the control system 505. In alternative embodiments utilizing wireless modes of communication between the control system 505, the driver 520, and/or the device 300, the wireless communication mechanisms may allow for similarly specific and direct communication between the individual components of the system 500.

For example, in the pictured embodiment, the processor 530 is operatively coupled to the sensors 370, 372 (and/or a communication module, as described below in relation to FIG. 6a) on the intravascular flow-modifying device 300 by way of a sensor cable or lead 540. In alternate embodiments, the processor 530 may be wirelessly coupled to the sensor 370 and/or the sensor 372 (and/or a communication module) on the intravascular flow-modifying device 300. Similarly, the processor 530 is shown operatively coupled to the at least one remote sensor 515 by way of a sensor cable or lead 545. In alternate embodiments, the processor 530 may be wirelessly coupled to the at least one remote sensor 515. Thus, in various embodiments, the controller 505 receives a sensor signal from the sensors 370, 372, and/or 515 and/or a communication module (and/or a communication module) either wirelessly or by way of sensor cables 540 and/or 545, and transmits control signals to the device 300 either wirelessly or by way of a command cable 550 linking the processor 530 to the driver 520 and/or a driver cable 555 linking the driver 520 to the device 300.

The blood pressure control system 500 may operate as a closed loop utilizing feedback from the sensors 370, 372, and/or 515, or as an open loop utilizing commands received from the user through the input device 527. The patient and/or treating physician may provide commands to the input device 527. The output device 525 may be used to display the sensor data/signal, the command signal, and/or the software and stored data contained in the memory 535. Thus, during the open loop operation of the system 500, the user may utilize some feedback from the sensors 370, 372, and/or 515, which may be displayed to the user on the output device 525, but the user may also operate the system 500 without any sensor feedback. Commands received by the input device 527 may directly influence the command signals issued by the processor 530 or may alter the software and related algorithms contained in the processor 530 and/or the memory 535.

In a closed loop, if the sensor 515 detects a reduction in cardiac output or systemic blood pressure, or if the sensor 370 detects a reduction in renovascular pressure, the control system 505 may generate an activation command signal to activate the device 300, thereby increasing renovascular perfusion such that the kidney 170 does not experience reduced blood flow (renal perfusion). When the sensor 515 or the sensor 370 detects the desired improvement or normalization of the sensed parameter (e.g., blood pressure), the control system 505 may generate a control command to deactivate or modify the flow restriction activity of the device 300.

In some embodiments, command signals generated by the processor 530 in response to user input from the input device 527 may override the command signals generated by the processor 530 in response to sensed data from the sensors 370, 372, and/or 515.

The processor 530 may contain information about the sensors 370, 372, and/or 515, such as what type of sensor it is (e.g., what the sensor detects, and how) and the location of the sensor (e.g., whether the sensor is located within the device 300, intravascularly, or outside the patient's body). Such information may be used by the processor 530 to select appropriate algorithms, lookup tables, and/or calibration coefficients stored in the processor 530 and/or the memory 535 for calculating the patient's appropriate physiological parameters. In addition, the processor 530 may contain information specific to the patient, such as, for example, the patient's age, weight, cardiovascular history, and diagnosis. This information may allow the processor 530 to determine patient-specific threshold ranges in which the patient's physiological parameter measurements should fall and to enable or disable additional physiological parameter algorithms, such as alarm threshold ranges for the output device 525 of the system 500. Moreover, the memory 535 may store such information for communication to the processor 530. By way of non-limiting example, the memory 535 may store the type and location of various sensors, the mechanism of action of various sensors, the proper algorithms to be used for calculating the patient's physiological parameters and/or alarm threshold values, the patient characteristics to be used for calculating the alarm threshold values, and the patient-specific threshold values to be used for monitoring the physiological parameters.

The processor 530 may be configured to calculate physiological parameters based on data inputted from the user through the input device 327 and the data received from the sensors 370, 372, and/or 515 relating to cardiovascular conditions. The processor may relay such information and calculations to the output device 525 for display to the user. As mentioned above, the output device may generate a visual, audible, or tactile warning to alert the user to sensed cardiovascular parameters that may require medical attention, including adjustment (e.g., activation or deactivation) of the device 300. In addition, the processor 530 may be connected to a network to enable the sharing of information with servers or other workstations (not shown).

The command signal generated by the processor 530 may be continuous, periodic, episodic, or a combination thereof, as dictated by an algorithm contained in the processor 530 and/or the memory 535. Continuous command signals include a constant pulse, a constant train of pulses, a triggered pulse, and a triggered train of pulses. Examples of periodic command signals include each of the continuous control signals described above which have a designated start time (e.g., the beginning of each minute, hour, or day) and a designated duration (e.g., 1 second, 1 minute, or 1 hour). Examples of episodic command signals include each of the continuous command signals described above which are triggered by a specific event, condition, or episode (e.g., activation by the user, an increase in sensed blood pressure above a certain threshold, etc.).

The processor 530 may be programmed to operate the device 300 in a range of power consumption modes, wherein the processor 530 issues continuous, periodic, episodic, and/or a combination thereof of command signals to the device 300, thereby controlling the amount of power to the device 300 and the activity of individual device components, such as, but not limited to, the sensors 370, 372, and/or 515 and the flow restrictor 360. In terms of operating in different power consumption modes, the sensors 370, 372 may be configured to operate in multiple modes that each consume a different amount of power. In all the embodiments described herein, each individual power consumption mode may correspond to using a different mix of sensors and/or a different data sampling regime. For example, in a high power consumption mode, one of or both the sensors 370, 372 may receive periodic command signals from the processor 530 to sense a particular characteristic only at certain interval for a limited duration. Depending on the current power consumption mode that the device 300 is operating in, one or more of the sensors may be de-energized to save power.

For example, in a high power consumption mode, the processor 530 may issue a continuous command signal to the sensors to sense various intravascular characteristics continuously. In a low power consumption mode, in contrast, the processor 530 may be programmed to issue periodic, episodic, and/or a combination of periodic and episodic command signals to the device 300, thereby minimizing the amount of activity of the sensors 370, 372 and/or the flow restrictor 360. In one example, the processor 530 may issue a periodic command signal regime directing the sensors to only sense a particular intravascular characteristic for 5 seconds every 60 seconds. In a low power consumption mode, the processor 530 may also selectively activate particular sensors without activating others. For example, if the upstream sensor 370 reports data confirming a stable cardiovascular state, the processor 530 may not direct the downstream sensor to detect anything.

The particular voltage, current, and frequency delivered to the device 300 may be varied in different power consumption modes as needed. For example, electrical energy can be delivered to the device 300 at a particular voltage, at a particular current, at a particular frequency, at a particular pulse-width, and at a particular combination of the foregoing to modulate the energy delivery to the device 300 depending upon the particular power consumption mode of the device 300 at any given time. Moreover, electrical energy can be delivered in a unipolar, bipolar, and/or multipolar sequence or, alternatively, via a sequential wave, charge-balanced biphasic square wave, sine wave, or any combination thereof depending upon the particular power consumption mode of the device 300 at any given time.

The processor 530 may select the mode of operation for the device 300 in real-time based on an analysis of the data obtained from the sensors 370, 372, and/or 515, or in response to input commands inputted into the input device 527 by a user. It should be understood that the various power consumption modes may comprise any of a variety of command signal regimes, provided certain modes permit the device 300 to consume less power and other modes direct the device 300 to consume more power.

The memory 535 may also store information for use in selection of a power consumption mode based on the data generated by sensors 370, 372, and/or 515 and/or the user inputs into the input device 327. For example, in some embodiments utilizing episodic command signal regimes, the memory 535 stores one or more data profiles that may be used to determine when the sensed data indicates that the device 300 should switch to a low power mode. A data profile may be an algorithm, table, or other representation of standard data to which the patient-specific data may be compared. If a match is detected between the patient-specific data and the relevant data profile, then the system 500 may switch to a low power mode of operation until some sensed trigger or episode causes the system to switch to another power consumption mode (e.g., to a high power consumption mode). Furthermore, the data profiles may identify which power consumption mode to use when a particular data profile is matched by the sensed data.

In some embodiments, the various power consumption modes may also be stored in the memory 535. For example, the memory 535 may include a listing of specific actions to be performed or not to be performed, or a list of components to be energized or de-energized while in a specific power mode. For example, if the sensors detect and report data conveying a normotensive cardiovascular state, and the normotensive data matches a normotensive data profile store on the memory 535, the system 500 may switch to a low power mode of operation during which neither the flow restrictor 360 nor the sensors 370, 372 are energized, or during which the sensors are energized on a periodic basis. Alternatively, in a hardware embodiment the various power modes may be incorporated into the hardware or firmware of the system.

FIG. 6a schematically shows the component parts of the intravascular flow-modifying device 300 in an expanded condition according to one embodiment of the present disclosure. The intravascular flow-modifying device 300 comprises an expandable support body 600 configured for insertion into a blood vessel and for stable implantation within the blood vessel. The support body 600 is shaped as a hollow, generally cylindrical tube that extends from the proximal end 340 to the distal end 350 of the device 300 and includes a main body portion 602 extending therebetween. The main body portion 602 houses the flow restrictor 360, the upstream sensor 370, and the downstream sensor 372. In addition, the main body portion 602 houses a driver 605 that is coupled to the flow restrictor and/or a microprocessor 610, a power supply 615 that may power various components of the device 300, and a communication module 620 that enables bidirectional communication between the device 300 and the control system 505 (and/or the remote sensor 515 shown in FIG. 5). Some embodiments may include at least one auxiliary sensor 625, which may be substantially similar in form and function to any of the sensors 370, 372, or 515. These individual components of the device 300 may be embedded within or disposed upon the expandable support body 600. In embodiments having individual components disposed upon the support body 600, individual components may be coupled to the support body 600 by any of a variety of attachment mechanisms, including, but not limited to, biologically compatible adhesive, welding, chemical bonding, and mechanical fasteners.

The support body 600 is configured to be an elongate, relatively flexible, cylindrical tube having an unexpanded condition and an expanded condition. Typically, the support body 600 has a structure that minimizes the risk of damage to individual components of the device 300 when the support body 600 is transformed between an unexpanded condition and an expanded condition. The flexible and expandable properties of the expandable support body 600 facilitates percutaneous delivery of the expandable support member, while also allowing the expandable support body 600 to conform to an intraluminal portion of a blood vessel (as illustrated in FIG. 3). In the expanded condition, the support body 600 has a generally circular cross-sectional shape for conforming to the generally circular cross-sectional shape of a blood vessel lumen. By conforming to the shape of a blood vessel lumen, the expanded configuration of the support body 600 facilitates movement of the blood flow therethrough while also maintaining lumen patency. In some embodiments, the support body 600 may be sized and configured for expansion, manipulation, and use within a renal vessel.

The structure of the expandable support body 600 may be, by way of non-limiting example, a mesh, a zigzag wire, a spiral wire, an expandable stent, or other similar configuration that defines a lumen 630 and allows the support body 600 to be collapsed and expanded intravascularly. The support body 600 may be fabricated from a self-expanding material biased such that the exterior surface of the support body 600 expands into contact with the vessel luminal wall upon expanding the device 300. Thus, the support body 600 may be comprised of a material having a high modulus of elasticity, including, for example, cobalt-nickel alloys (e.g., Elgiloy), titanium, nickel-titanium alloys (e.g., Nitinol), cobalt-chromium alloys (e.g., Stellite), nickel-cobalt-chromium-molybdenum alloys (e.g., MP35N), graphite, ceramic, stainless steel, and hardened plastics. The expandable support body 600 may also be made of a radiopaque material or include radiopaque markers (e.g., radiopaque markers 388, as shown in FIG. 3) to facilitate the fluoroscopic visualization of the intravascular positioning and placement of the device 300.

The support body 600 may include at least one therapeutic agent for eluting into the vascular tissue and/or blood stream. The therapeutic agent may be capable of counteracting a variety of systemic and local pathological conditions including, but not limited to, hypertension, hypotension, thrombosis, stenosis, and inflammation. Accordingly, the therapeutic agent may include at least one of an anti-hypertensive, an anti-hypotensive agent, an anticoagulant, an antioxidant, a fibrinolytic, a steroid, an antiapoptotic agent, and/or an anti-inflammatory agent. In some embodiments, the therapeutic agent may be capable of treating or preventing other diseases or disease processes such as microbial infections and heart failure. In these instances, the therapeutic agent may include an inotropic agent, a chronotropic agent, an anti-microbial agent, and/or a biological agent such as a cell, peptide, or nucleic acid. The therapeutic agent may be linked to the interior or exterior surface of the support body 600, embedded and released from within polymer materials, such as, by way of non-limiting example, a polymer matrix, or surrounded by and released through a carrier member (not shown) that is associated with the support body 600.

In some embodiments, the expandable support body 600 includes an insulative material 635 for isolating blood flow through the vessel 12 from any electric current flowing through the device 300. Thus, the insulative material 635 may serve as an electrical insulator, separating electrical energy from the surrounding blood flow and tissue and facilitating efficient delivery of the electrical energy to individual components of the device 300. The insulative material 635 generally has a low electrical conductivity and a non-thrombogenic surface. The insulative material 635 may include materials such as, by way of non-limiting example, PTFE, ePTFE, silicone, silicone-based materials, elastomeric materials, an ultraviolet cure or heat shrink sleeve, polyethelene, Nylon™, and the like. In the pictured embodiment, the insulative material 635 is disposed around the support body 600 and extends along the entire exterior and interior length of the body 600. Alternatively, the insulative material 635 may be attached to select portions of the device 300, including, but not limited to, the expandable support body 600, the sensors 370, 372, and the power supply 615. Additionally or alternatively, the insulative material 635 may be disposed about the luminal surface of the expandable support body 600, the non-luminal surface of the support body 600, or may be wrapped around both the luminal and non-luminal surfaces. The insulative material may be attached around the entire circumference of the expandable support body 600 or, alternatively, may be attached in pieces or interrupted sections to allow the expandable support body 600 to more easily expand and contract.

In some embodiments, at least a portion of the device 300, including the support body 600 and/or other individual components of the device 300, may optionally include a layer (not shown) of biocompatible material. The layer of biocompatible material may be synthetic such as Dacron® (Invista, Wichita, Kans.), Gore-Tex® (W. L. Gore & Associates, Flagstaff, Ariz.), woven velour, polyurethane, or heparin-coated fabric. Alternatively, the layer of biocompatible material may be a biological material such as, by way of non-limiting example, bovine or equine pericardium, peritoneal tissue, an allograft, a homograft, patient graft, or a cell-seeded tissue. The biocompatible layer may cover either the luminal surface of the expandable support body 600, the non-luminal surface of the support body 600, or may be wrapped around both the luminal and non-luminal surfaces. The biocompatible layer may be attached around the entire circumference of the expandable support body 600 or, alternatively, may be attached in pieces or interrupted sections to allow the expandable support body 600 to more easily expand and contract.

The flow restrictor 360 is disposed within the expandable support body 600 such that the flow constrictor 360, when activated, may partially occlude the vessel lumen. The flow restrictor 360 may be configured to include any of a variety of forms and mechanisms of action, provided that the flow restrictor can partially occlude blood flow through the device 300 and thereby create an area of artificially increased blood pressure immediately upstream of the device 300 which modulates the activity of baroreceptors in the vicinity.

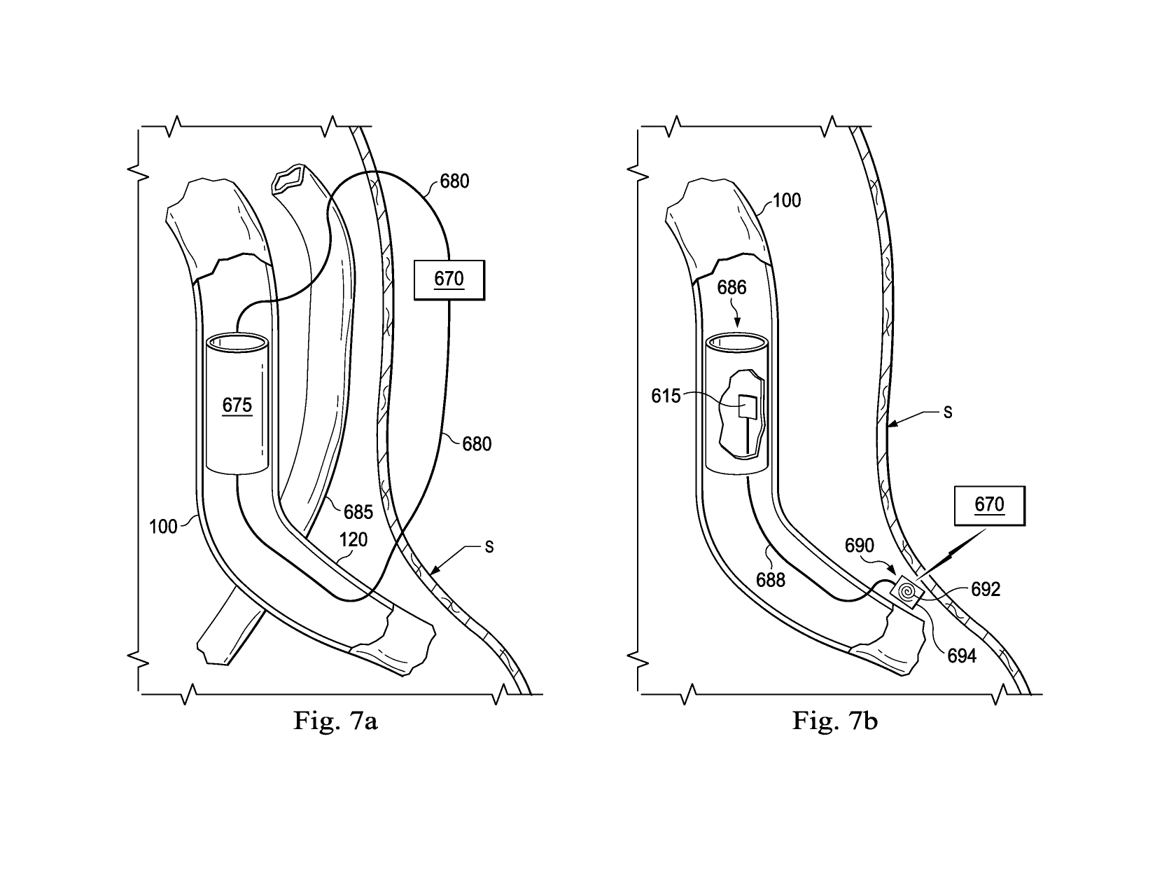

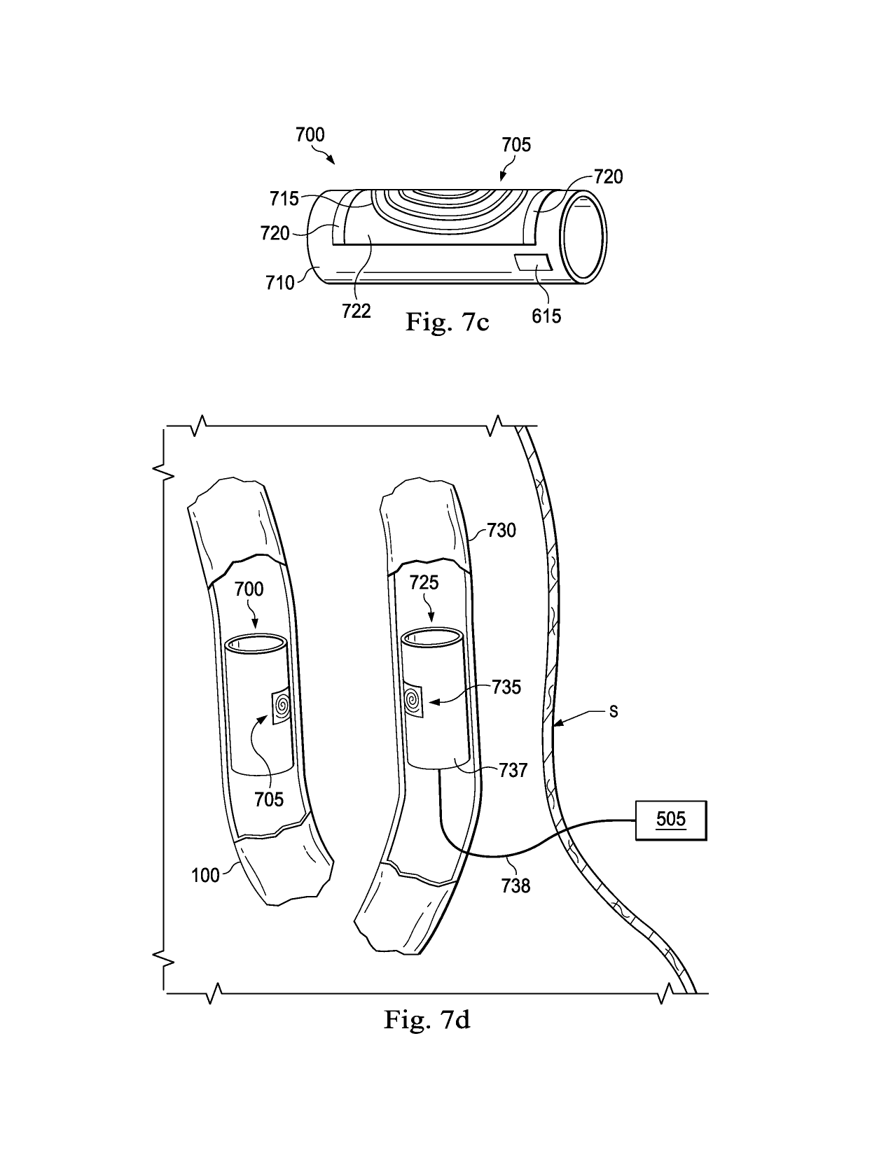

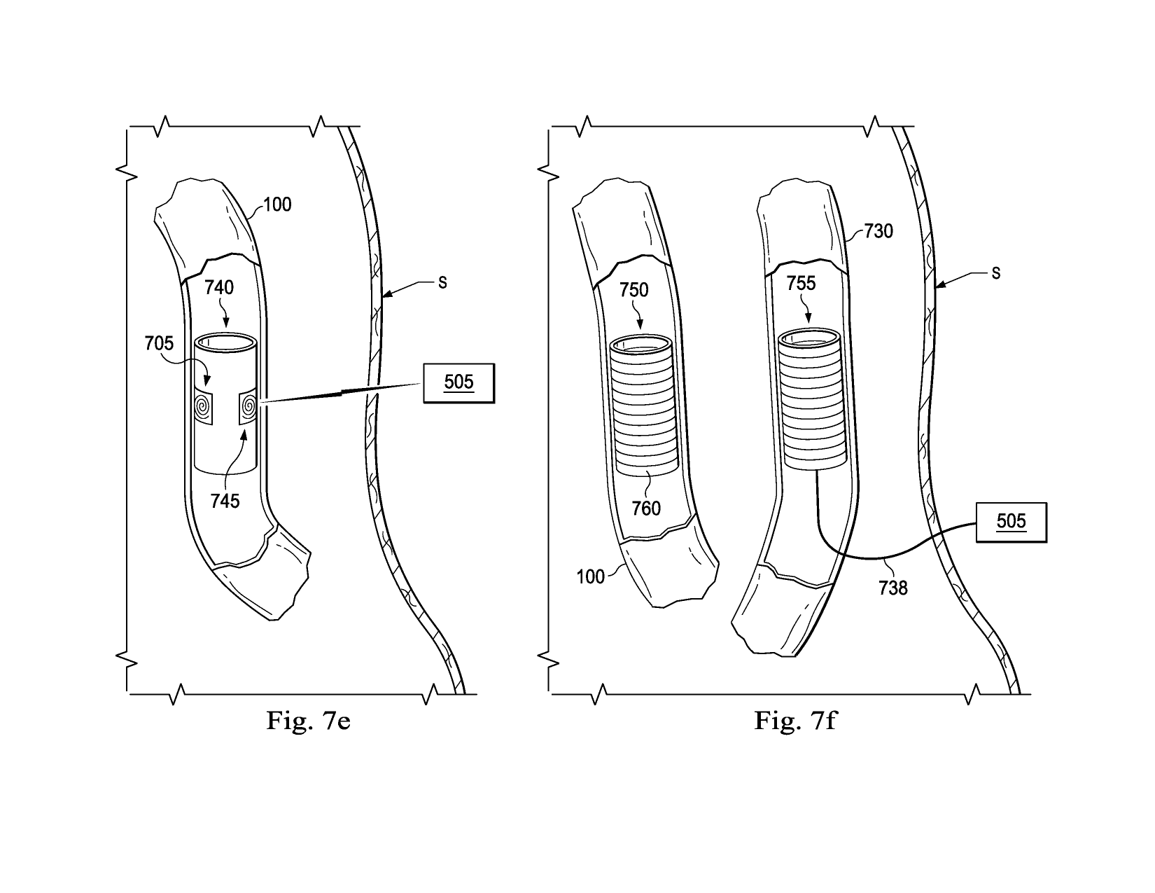

The driver 605 comprises an actuator apparatus coupled to the flow restrictor 360 such that the driver 605 may impel the flow restrictor 360 to change from an inactivated condition to an activated condition capable of restricting flow through the lumen 630 of the support body 600. For example, upon receiving an activation signal from the microprocessor 610, the driver 605 actuates or activates the flow restrictor 360, moving it from an inactive position or a less active position to a more active position, thereby increasing the degree of occlusion within the support body 600 and the vessel lumen. Conversely, upon receiving a deactivation signal from the microprocessor 610, the driver 605 deactivates the flow restrictor 360, moving it from a more active position to a less active position, thereby decreasing the degree of occlusion within the support body 600 and the vessel lumen. Various specific embodiments of a driver may be described below in relation to FIGS. 7-15b. By way of non-limiting example, the driver may comprise or be coupled to any of an actuating rod, a helical coil, a motor, a piston, and/or a pump.

As mentioned above in relation to FIGS. 3 and 5, exemplary sensors 370, 372 may include, without limitation, ultrasonic sensors, flow sensors, thermal sensors, such as thermocouples, thermistors and infrared sensors, pressure sensors, electrical contact sensors, conductivity and/or impedance sensors, electromagnetic detectors, fluid flow sensors, electrical current sensors, tension sensors, chemical or hormonal sensors (capable of detecting the concentration or presence/absence of various gases, ions, enzymes, proteins, metabolic products, etc.), and pH sensors. The expandable support body 600 may contain any of a variety of sensor types within a single embodiment. As a result, the device 300 may be capable of simultaneously examining a number of different characteristics of the blood and surrounding tissue, the surrounding environment, and/or the device itself within the body of a patient, including, by way of non-limiting example, vessel wall temperature, blood temperature, device temperature, fluorescence, luminescence, flow rate, and flow pressure.

The sensors 370, 372 may comprise raised components or flat components on the surface of the support body 600. The sensors 370, 372 may be located at any position along the length of the body 600, provided that the sensor 370 is positioned upstream to the flow restrictor 360, and the sensor 372 is positioned downstream to the flow restrictor 360. The sensors may be coupled to the expandable support body using any of a variety of known connection methods, including by way of non-limiting example, welding, biologically-compatible adhesive, and/or mechanical fasteners. For example, in one embodiment, the sensors 370, 372 may be adhesively bonded to the body 600 using Loctite 3311 or any other biologically compatible adhesive. In some embodiments, the sensors may be integrally formed with the support body 600. For example, in some embodiments, at least one sensor 370, 372 may be comprised of flexible circuits integrated into the support body 600. The flexible circuit may be comprised of polymer thick film flex circuit that incorporates a specially formulated conductive or resistive ink that is screen printed onto the flexible substrate to create the sensor circuit patterns. This substrate is then adhered to a surface of the support body 600 or integrated with the support body 600.

In addition to the upstream sensor 370 and the downstream sensor 372, the device 300 may include any number of ancillary sensors 625 positioned on the exterior, vessel wall-contacting surface of the support body 600. Except for their position, the ancillary sensors may be configured to be substantially similar to sensors 370, 372. Exemplary ancillary sensors 625 include, without limitation, ultrasonic sensors, flow sensors, thermal sensors, blood temperature sensors, electrical contact sensors, conductivity sensors, electromagnetic detectors, chemical or hormonal sensors, pH sensors, and infrared sensors. For example, in one embodiment the ancillary sensor 625 may comprise a thermal sensor positioned on the exterior vessel wall-contacting surface of the support body 600, thereby permitting the sensor 625 to measure a characteristic of the vessel wall (e.g., temperature) while simultaneously the sensors 370, 372 may measure a cardiovascular characteristic within the vessel lumen.

In some embodiments, each sensor 370, 372, and/or 625 includes sensor cables (not shown) coupling the sensor to at least the microprocessor 610 and/or the communication module 620. In alternate embodiments, several sensors may be coupled to the microprocessor 610 and/or the communication module 620 using one or more shared sensor cables. In alternate embodiments, the sensors may communicate with the microprocessor 610 and/or the communication module 620 via any of a variety of wireless means.

The communication module 620 is configured to relay information, such as command signals from the processor 530 and sensed data from the sensors 370, 372, between the device 300 and the control system 505. The communication module 620 may contain transmitter circuitry and receiver circuitry that together carry out the bidirectional communication with the control system 505. The communication module 620 may cooperate with the control system 505 to actively control power transmission, activation energy, power mode, and/or an activation protocol. In some embodiments, the communication module may operate in a closed loop fashion by actively controlling power transmission, activation energy, power mode, and/or an activation protocol for the device 300 without receiving instructions from the control system 505. Instead, the communication module 620 may communicate internally with the sensors 370, 372, the power supply 615, the microprocessor 610, and/or the driver 605 to operate the device 300. In some embodiments, the communication module 620 may operate in both an open loop and closed loop fashion to operate the device 300.

In some embodiments, the communication module is coupled to the control system 505 via sensor cables 540, as described above in relation to FIG. 5. In other embodiments, the communication module 620 is coupled to the control system 505 via wireless means. In such embodiments, as illustrated in FIG. 6b, the communication module 620 may include an antenna 640 and a transceiver 645 coupled to the antenna 640. The antenna 640 is capable of sending signals to the control system 505 and receiving signals from the control system 505. In some embodiments, the signals are transmitted and received at Radio Frequencies (RF).

The device 300 includes a microprocessor 610 that is coupled to the communication module 620. Specifically, the microprocessor may be coupled to the transceiver 645. Based on the output of the transceiver 645 (i.e., the input received from the control system 505), the microprocessor runs firmware 650, which is a control program, to operate control logic 655, which is the dedicated software code that is written to operate the device 300. In embodiments configured for wireless communication, the control logic 655 may include digital circuitry that is implemented using a plurality of transistors, for example Field Effect Transistors (FETs). In the pictured embodiment, the firmware 650 and the control logic 655 are integrated into the microprocessor 610. In alternate embodiments, the firmware 650 and/or the control logic 655 may be implemented separately from the microprocessor 610. As mentioned above, the driver 605 controls the flow restrictor 630 upon receiving an output signal from the microprocessor 610.

The power supply 615 is configured to provide power to the other components of the device 300, and may include power circuitry 660 and a rechargeable power source 665. In some embodiments, the power source 665 includes a battery that may be coupled to an external power supply via a cable (not shown). In other embodiments, the power source 665 includes a receiving coil that is part of a transformer (not shown). In that case, the transformer also includes a remote charging coil that is positioned external to the power source 665 and inductively coupled to a receiving coil of the power source 665. Thus, as described in more detail below with reference to FIGS. 7b-7f, the power source 665 may obtain energy from the inductive coupling between a receiving coil of the power source 665 and the remote charging coil. In alternate embodiments, the power source 665 includes both a battery and a receiving coil.

In alternate embodiments, the power source 665 utilizes a piezoelectric mechanism, such as, by way of non-limiting example, a piezoelectric crystal and a piezoelectric wire, to generate RF energy. In alternate embodiments, the power source 665 includes both a battery and a piezoelectric crystal. In some embodiments, the power source 665 utilizes an amplifier (not shown) to amplify the RF signal generated wirelessly through either an inductive coupling or a piezoelectric mechanism. In some embodiments, the power source 665 utilizes an AC/DC converter to supply power the individual components of the device 300.

In any case, the power source 665 must provide a sufficient amount of power to meet the needs of the device 300 and must be small enough to fit within the slim profile of the support body 600 that is preferred clinically. The power source 665 may, but need not be, rechargeable. Whether or not the power source is rechargeable, given the relatively significant power requirements of the various on-board sensors 370, 372, and the relatively limited amount of power available in a power source small enough to be integrated into the device 300, prudent power management must be employed to enable the device 300 to operate without necessitating that the device 300 be removed from the vasculature for replacement, and/or, if applicable, recharging of the power source.

This challenge may be overcome by a combined power conservation approach that involves power consumption mode protocols orchestrated by the user, the control system 505 (as described above in relation to FIG. 5), and/or the device 300 itself. The microprocessor 610, the sensors 370, 372, and the power supply 615 may cooperate to direct the device 300 through a variety of power consumption modes in a substantially identical fashion as that described above in relation to the operation of the control system 505. In response to sensed cardiovascular data by the sensors 370, 372, the microprocessor 610 and the power supply 615 may cooperate to deliver varying amounts of power to the flow restrictor 360 and/or the sensors 370, 372, thereby conserving power when possible. Thus, the microprocessor 610 may lead the device 300 through a variety of power consumption modes during which the sensors 370, 372 and the flow restrictor 360 function in a variety of active and inactive states suited to the existing cardiovascular conditions of the patient, thereby conserving power when appropriate. This power consumption mode protocol may prolong the service life of the power supply 615.

The device 300, and the various components thereof, may be manufactured from a variety of materials, including, by way of non-limiting example, plastics, polytetrafluoroethylene (PTFE), polyether block amide (PEBAX), thermoplastic, polyimide, silicone, elastomer, shape memory materials, metals, such as stainless steel, titanium, shape-memory alloys such as Nitinol, polymers, composite materials, and/or other biologically compatible materials. In addition, the device 300 may be manufactured in a variety of lengths, diameters, dimensions, and shapes to accommodate a variety of applications. The wall of the support body 600 is configured to be sufficiently thin so as not to significantly restrict blood flow through the unactivated device 300. The outer diameter of the device 300 may be varied so as to fit within a particular blood vessel and to adapt to different blood vessel sizes. Similarly, the length of the device 300 may be varied according to anatomical and applicational need. For example, in one embodiment the support body 600 may be manufactured to have length of about in the range of 2-5 cm. In another embodiment, the support body 600 of the device 300 may be manufactured to have a transverse dimension or diameter of about 5-8 mm, thereby permitting the device to be configured for insertion into the renal vasculature of a patient.

With general reference to FIGS. 7a-9, schematic illustrations of specific embodiments of the intravascular flow-modifying device 300 utilizing various power supply arrangements are shown. In most instances, each intravascular flow-modifying device is configured to restrict intravascular flow when the device is activated and powered. Conversely, when the device is inactivated or unpowered, the device is configured to allow as much flow as possible through the device while still maintaining an expanded condition within the vessel lumen. A remote or local energy source may be physically or remotely coupled to the intravascular flow-modifying device to provide energy to the power supply (e.g., 615) of the device.

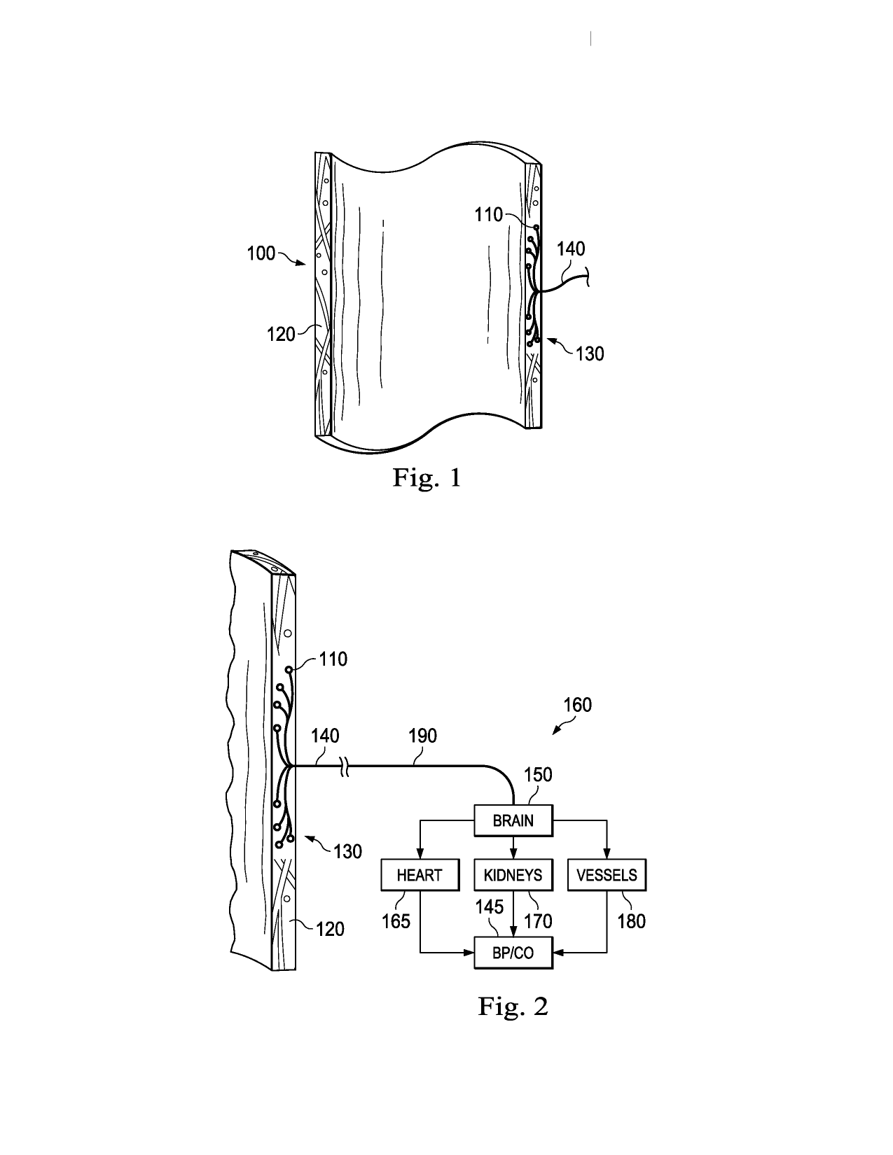

The design, function, and use of these specific embodiments, in addition to the control system 505 and the driver 520, are the same as described with reference to FIG. 6, unless otherwise noted or apparent from the description. In addition, any anatomical features illustrated in FIGS. 7a-9 are the same as discussed with reference to FIGS. 1 and 2, unless otherwise noted. In each embodiment, the connections between the individual components of the device (e.g., the microprocessor, the driver, the communication module, the power supply, the sensors, and/or the flow constrictor) may be physical (such as, by way of non-limiting example, wires, tubes, cables, etc.) or remote (such as, by way of non-limiting example, wireless transmitter/receiver, inductive coupling, magnetic coupling, etc.). For physical connections, the connection may travel intra-arterially, intravenously, subcutaneously, or through other natural tissue paths.

As stated above, an energy source may be physically or remotely coupled to the intravascular flow-modifying device to provide energy to the device. As shown in FIG. 7a, according to one embodiment of the disclosure, an external energy source 670 may be directly coupled to an intravascular flow-modifying device 675 positioned within the blood vessel 100 using an electrical cable or lead 680. The electrical cable 680 may travel down a length of the blood vessel 100 before emerging through the vessel wall 120 to exit the patient's body through the skin S (e.g., at the insertion site for the device 675). In alternate embodiments, the cable 680 may exit through the vessel wall 120 to enter an adjacent vessel 685 before eventually exiting the patient's body through the skin S. In some embodiments, the external energy source may be coupled to and controlled by the control system 505 and/or the driver 520 (shown in FIG. 5).

In addition to physical power connections, an energy source may be wirelessly coupled to the device to provide a remote means of supplying energy to the device. FIGS. 7b-7f schematically illustrate various types of wireless energy transmission arrangements for use with any of the intravascular flow-modifying devices described herein. Various types of energy may be supplied to the power source 625. The energy types may include, for example, radio frequency (RF) energy, X-ray energy, microwave energy, acoustic or ultrasound energy such as focused ultrasound or high intensity focused ultrasound energy, light energy, electric field energy, magnetic field energy, combinations of the same, or the like. As mentioned above in relation to FIGS. 5 and 6, energy may be delivered to the various components of the device continuously, periodically, episodically, or a combination thereof depending upon the particular power consumption mode of the device at any given time.

FIG. 7b illustrates an intravascular flow-modifying device 686 positioned within the vessel 100. The device 686 includes an electrode cable or lead 688 that couples the device 686 to a receiving coil assembly 690, which may be implanted extravascularly within subcutaneous tissue (as shown) or intravascularly near the skin surface S (not shown). The receiving coil assembly 690 may include a receiving coil 692 disposed on a flexible substrate 694. The receiving coil 692 may receive energy from an external energy source 670, such as, by way of non-limiting example, a transmitting coil, and then transmit the energy to through the cable 688 to the power supply 615 of the device 686.

FIG. 7c illustrates an intravascular flow-modifying device 700 configured for wireless power acquisition according to one embodiment of the present disclosure. The device 700 includes a receiving coil assembly 705 wrapped about an external surface 710 of the device 700. The receiving coil assembly 705 may be integrally formed with the device 700, or may be movably attached to the device 700 to permit free expansion of the receiving coil assembly 705 with expansion of the device 700. The receiving coil assembly 705 may be shaped in the form of a semi-cylinder as shown or in the form of a cylindrical sleeve (not shown). The receiving coil assembly includes a receiving coil 715 that may be connected to at least one (optional) electrode pad 720. The coil 715 and the electrode pads 720 comprise a conductive metal disposed on a flexible substrate material 722. The metal may be laminated onto the substrate material 722, or the substrate 722 may be chemically etched to define the coil 715 and the electrode pads 720. The coil 715 transfers received energy to the power supply 615 of the device 700. In some embodiments, the energy transfer may be transferred through the electrode pad 720 to the power supply 615 or other components of the device 700 (not shown).

FIG. 7d illustrates the intravascular flow-modifying device 700 in a wireless transmission arrangement with an intravascular transmitter device 725 according to one embodiment of the present disclosure. The device 700 is shown positioned in the vessel 100, which contains the baroreceptors of interest. A transmitting device 725 may be positioned in an adjacent vessel 730 that lies in close proximity to the vessel 100. In the pictured embodiment, the transmitting device 725 includes a coil assembly 735, which is similar to the construction and arrangement of assembly 705 disposed on device 700 as described previously, disposed on a stent-like tubular support structure 737. The transmitting device 725 and the flow-modifying device 700 are positioned and anchored within their respective vessels such that their coil assemblies, 735 and 705, respectively, are arranged “face-to-face.”

In alternate embodiments, the transmitting device is implanted in the subcutaneous tissue instead of within a vessel. In such embodiments, the transmitting coil assembly may be disposed on a differently shaped support structure than the tubular support structure 737.

The coil assembly 735 may emit an RF or other electromagnetic signal picked up by the coil assembly 705 on the intravascular flow-modifying device 700. In some embodiments, the transmitting coil assembly 725 may be under the control of the user and/or the control system 505. In some embodiments, the coil assembly 735 on the transmitting device 725 may act as an antenna to wirelessly receive command signals and energy from the control system 505. The coil assembly 735 on the transmitting device 725 may act as an antenna to wirelessly receive command signals from the control system 505, or may be operably coupled to the control system 505 (not shown) via physical cables or leads 738 which travel down the vessel 730 through the skin S. The transmitting device 725 is preferably disposed in a venous vessel to reduce the risk of thromboembolism and stroke.

FIG. 7e illustrates an intravascular flow-modifying device 740 in a wireless transmission arrangement according to one embodiment of the present disclosure. The device 740 is shaped and configured substantially identical to the device 700 except for the differences noted herein. The device 740 is shown positioned in the vessel 100, which contains the baroreceptors of interest. The device 740 includes a transmitter coil assembly 745, which is positioned on the external surface of the device 740 opposite to the receiver coil assembly 705. The transmitter coil assembly is similar to the construction and arrangement of assembly 705. The substantially “planar” receiver coil assembly 705 and the transmitter coil assembly 745 are positioned on the device 740 such that the assemblies are arranged “face-to-face.” In some embodiments, the transmitter coil assembly 745 may be under the control of the user and/or the control system 505. In some embodiments, the transmitter coil assembly 745 may be under the control of the microprocessor 610. The transmitter coil assembly 745 may emit an RF or other electromagnetic signal picked up by the receiver coil assembly 705.

FIG. 7f illustrates an intravascular flow-modifying device 750 in a wireless transmission arrangement according to one embodiment of the present disclosure. The device 750 includes an expandable support body 760 shaped and configured as a helical receiver coil. The device 750 is shown positioned in the vessel 100, which contains the baroreceptors of interest. A helical transmitter coil 755 may be positioned in the adjacent vessel 730 that lies in close proximity to the vessel 100. In the pictured embodiment, the helical transmitter coil 755 is similar to the construction and arrangement of the support body 760. The helical transmitter coil 755 and the helical flow-modifying device 750 are positioned and anchored within their respective vessels such that their coil axes are substantially aligned and/or are substantially parallel. The helical transmitter coil 755 may emit an RF or other electromagnetic signal picked up by the helical support body 760 of the intravascular flow-modifying device 700.

In some embodiments, the helical transmitter coil 755 may be under the control of the user and/or the control system 505. In some embodiments, the helical transmitter coil 755 may act as an antenna to wirelessly receive command signals and energy from the control system 505. The helical transmitter coil 755 may act as an antenna to wirelessly receive command signals from the control system 505, or may be operably coupled to the control system 505 (not shown) via physical cables or leads 738 which travel down the vessel 730 through the skin S. The helical transmitter coil 755 is preferably disposed in a venous vessel to reduce the risk of thromboembolism and stroke. Transmissions between the helical transmitter coil 755 and the helical support body 760 may be used to power the device 750. For example, as current is run through the helical transmitter coil 755, which may be coupled to the control system 505 via the cable 738, an electromagnetic field may be produced that induces a current in the helical support body 760. Such induced current may be harnessed by the power supply 615 (not shown) within the device 750 to charge the power supply 665 (not shown) or to directly power other individual components of the device 750. The size of the coils and the number of turns in each helical structure may determine the amount of energy delivered.

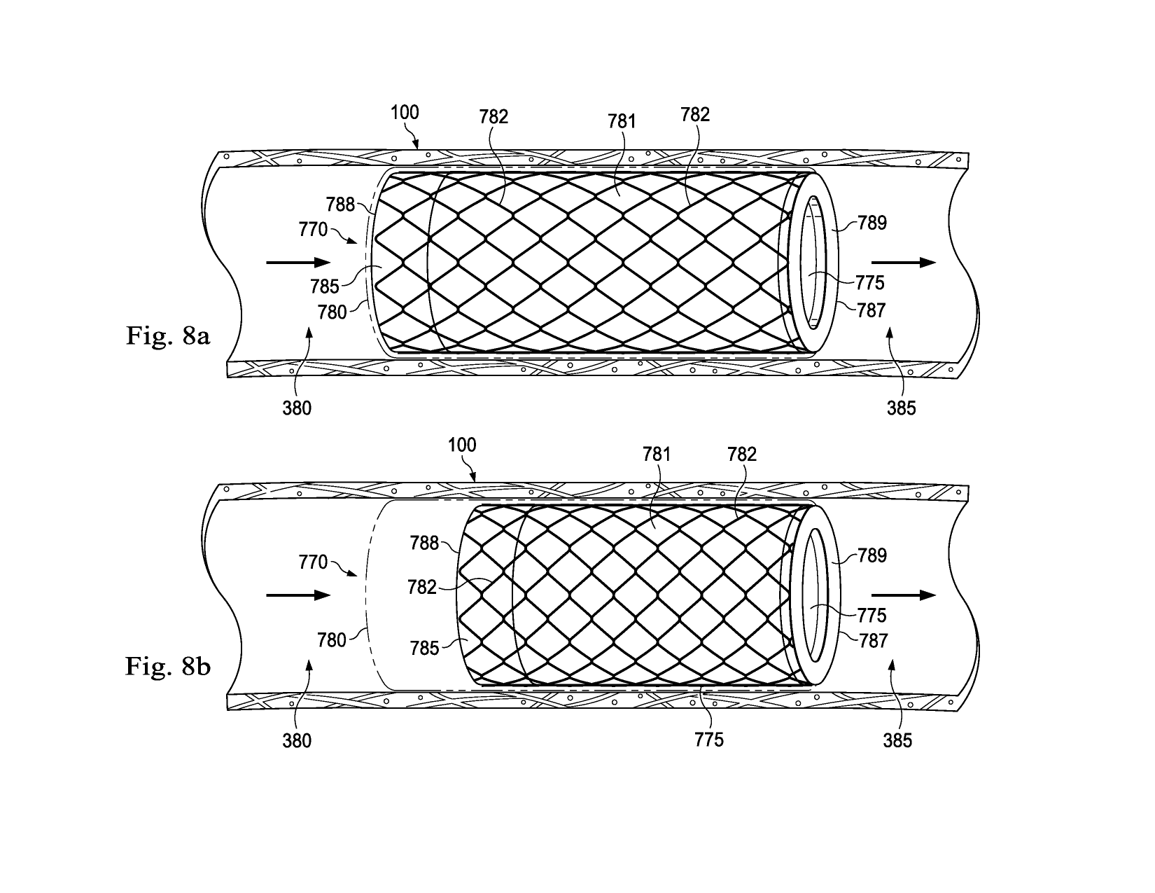

In alternate embodiments, as shown in FIGS. 8a-9, the intravascular flow-modifying device itself may be shaped and configured to generate energy in cooperation with the cardiovascular activity within the patient's body.

For example, FIG. 8a illustrates an intravascular flow-modifying device 770 according to one embodiment of the present disclosure. The device 770 is positioned within the vessel 100 such that blood flows from the upstream area 380, through a lumen 775 of the device 770, and into the downstream area 385. In this embodiment, the vessel 100 comprises an arterial vessel. The device 770 may include a hollow, cylindrical generator 775 housed within a hollow, cylindrical support body 780. The generator 775 includes a central body portion 781, a toroidal ring 785, and a toroidal ring 787. The central body portion 781 comprises a spring-like elongate, hollow cylinder formed of a plurality of electrically conductive wires 782. The ring 785 and the ring 787 are disposed at proximal and distal ends 788, 789, respectively, of the device 770. In the pictured embodiment, the ring 785 comprises an annular mass that is shaped and configured to have significantly more mass than the ring 787. The device 770 is anchored within the vessel 100 in the region of the ring 787. The rings 785, 787 may be magnetized, and may be formed of any of a variety of biocompatible materials, including, by way of non-limiting example, magnetic, ferromagnetic, paramagnetic, and/or non-magnetic materials.

The intravascular flow-modifying device 770 employs the principle of variable distance capacitance to generate energy, wherein the body portion 781 comprises a variable distance capacitor. As the patient's heart contracts, a pulse of blood contacts the proximal end 788 before travelling through the lumen 775 of the device 770. As shown in FIG. 8b, the proximal end 788 may be shaped and configured such that when the blood contacts the proximal end 788, the ring 785 is shifted toward the portion 787, thereby compressing the body portion 781 within the support body 780 and causing the conductive wires 782 to move closer to one another. As the patient's heart expands in preparation for the next beat, the decrease in intra-arterial pressure allows the body portion 781 to re-expand and the portion 785 to shift away from the portion 787. With each beat of the patient's heart, this cycle of compression and expansion of the body portion 781 sequentially repeats to transform kinetic energy into electrical energy (or current) within the body portion 781. The generated current may be harnessed by the power supply 615 (not shown for the sake of simplicity) within the device 770 to charge the power supply 665 (not shown for the sake of simplicity) or to directly power other individual components of the device 770.

FIG. 9 illustrates another intravascular flow-modifying device 790 shaped and configured to generate energy in cooperation with the cardiovascular activity within the patient's body according to one embodiment of the present disclosure. The device 790 includes a lumen 791 that contains a proximal fluid area 792, a distal fluid area 793, a flow restrictor 360, and a generator device 794. The device 790 includes a proximal end 795 and a distal end 796. The device 790 is positioned within the vessel 100 and the generator device is operatively disposed within the lumen 791 such that blood flows from the upstream area 380, through the proximal fluid area 792, through the generator device 794, through the distal fluid area 793, and into the downstream area 385. The generator device 794 may comprise an electrical generator that, in general, utilizes the mechanical energy associated with the movement of blood through the generator device 794 to generate electricity for powering the device 790. As fluid flows from the proximal fluid area 792 to the distal fluid area 793 through the generator device 794, electrical energy is generated.

The present disclosure contemplates the use of any suitable generator device 794 for use within the device 790 to accommodate particular needs. For example, the generator 794 may comprise a turbine mechanism that, in response to the propulsion of blood through the generator device 794 generated by the patient's own cardiovascular system (e.g., cardiac and vascular contractions and/or blood pressure changes), converts the kinetic energy of the blood into electric energy to charge the power supply 615 (not shown). In particular, as blood flows through the turbine mechanism, the turbine mechanism may be configured to rotate a conductive coil through a magnetic field created by opposing magnetic structures (e.g., magnetic rings located at the proximal and distal ends 795, 796, respectively, of the device 790) to induce an electric current in the conductive coil. The generated current may be harnessed by the power supply 615 (not shown) within the device 790 to charge the power supply 665 (not shown) or to directly power other individual components of the device 790.

In addition, micro-electrical-mechanical systems (MEMS) technology may provide various generators 794 for use in embodiments of the current disclosure. In some embodiments, the flow restrictor 360 of the device 790 may function as the generator device or may be integrally coupled to the generator device.

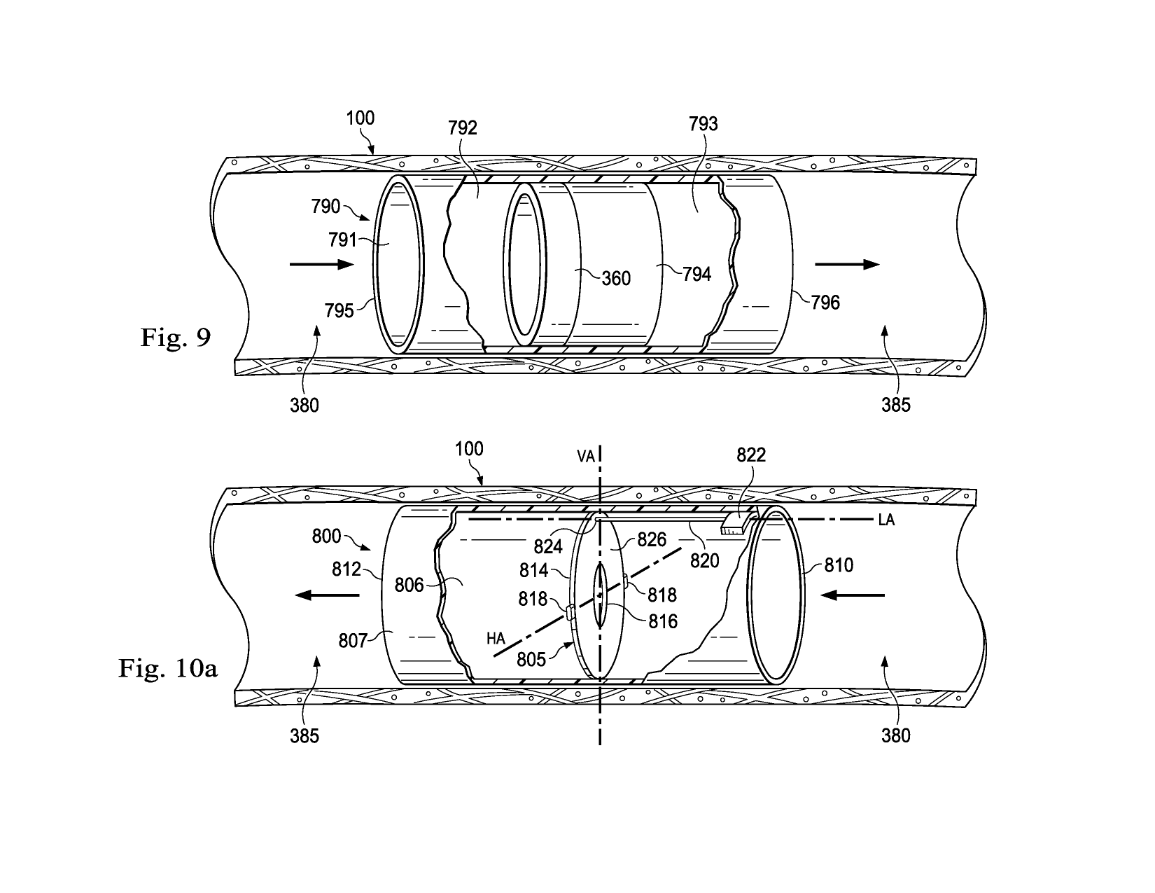

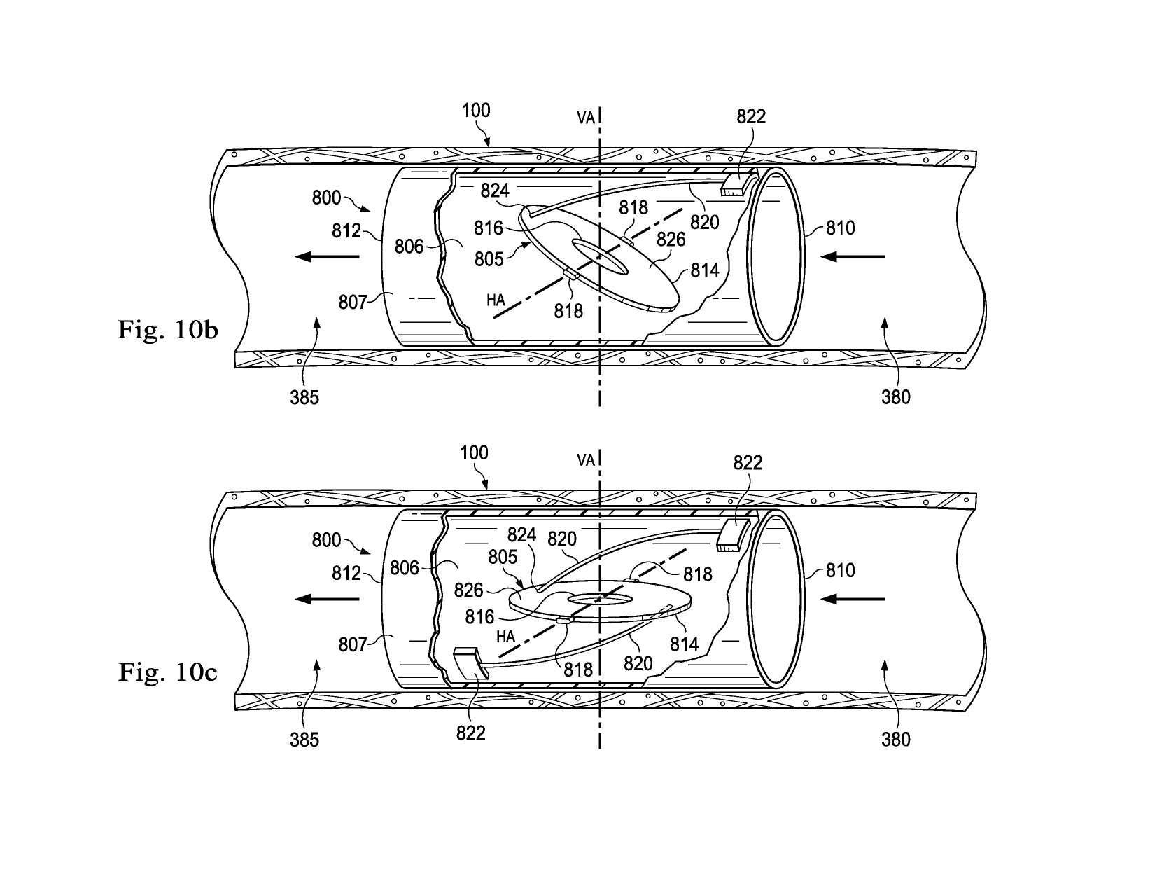

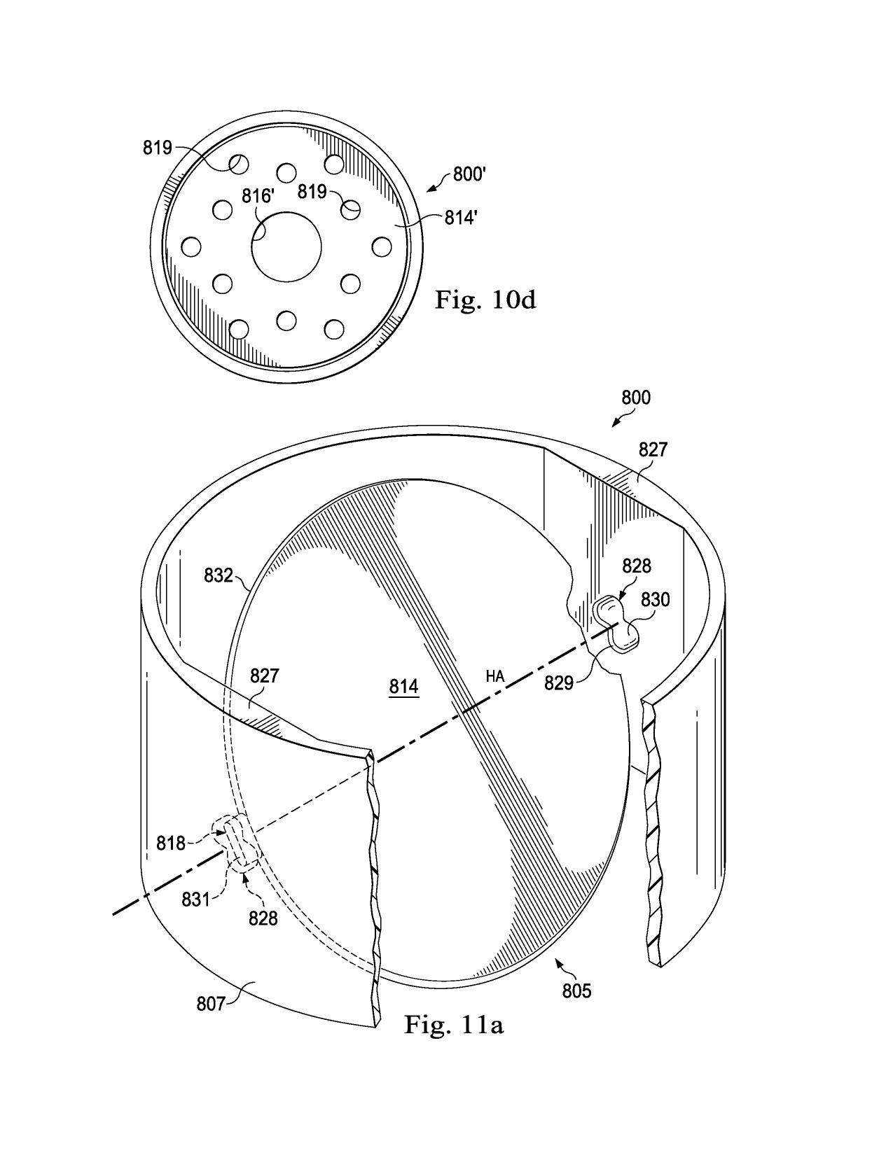

With general reference to FIGS. 10a-15b, schematic illustrations of specific embodiments of the intravascular flow-modifying device 300 are shown. As mentioned above, in general, each embodiment of the present disclosure is configured to restrict intravascular flow when the device is activated and powered. Conversely, when the device is inactivated or unpowered, the device is configured to allow as much flow as possible through the device while still maintaining an expanded condition within the vessel lumen. Specifically, each activated intravascular flow-modifying device indirectly modulates the baroreceptor system by restricting flow and creating back pressure upstream of the device, thereby artificially increasing the blood pressure upstream of the device to affect the baroreceptor system (either by deforming the vessel wall located immediately upstream of the intravascular flow-modifying device to activate baroreceptors and/or by increasing intrarenal perfusion and pressure to decrease baroreceptor-mediated sympathetic activity).

The design, function, and use of these specific embodiments, in addition to the control system 505 and the driver 520, are the same as described with reference to device 300 in FIG. 6a, unless otherwise noted or apparent from the description. In addition, any anatomical features illustrated in FIGS. 10a-15b are the same as discussed with reference to FIGS. 1 and 2, unless otherwise noted. In each embodiment, the connections between the individual components of the device (e.g., the microprocessor, the driver, the communication module, the power supply, the sensors, and/or the flow constrictor) may be physical (such as, by way of non-limiting example, wires, tubes, cables, etc.) or remote (such as, by way of non-limiting example, wireless transmitter/receiver, inductive coupling, magnetic coupling, etc.). For physical connections, the connection may travel intra-arterially, intravenously, subcutaneously, or through other natural tissue paths.

FIGS. 10a-10c illustrate an intravascular flow-modifying device 800 positioned within the vessel 100. The device 800 includes a flow restrictor 805 positioned centrally within a lumen 806 of an elongate, hollow, cylindrical support body 807. The device 800 includes a proximal end 810 and a distal end 812. The device 800 is shaped and configured for intravascular placement in a vessel such that the proximal end 810 is positioned upstream to the distal end 812, and blood flows from the intravascular area 380 proximal to the device 800, through proximal end 810, through the flow restrictor 805, and out the distal end 812 into the intravascular area 385 distal to the device 800. The flow restrictor 805 includes a pivotable disc 814 having a central aperture 816 and side tabs 818. The side tabs 818 pivotably anchor the disc 814 within the support body 807 such that the disc 814 may pivot from an active position (as shown in FIG. 10a) to a less active (as shown in FIG. 10b) or inactive position (as shown in FIG. 10c).

The aperture 816 permits blood flow through the device 800 even when the flow restrictor 805 is in an active condition. In some embodiments, the disc may include several perforations or apertures to permit sufficient blood flow through the device 800 even when the flow restrictor 805 is in an active condition. For example, FIG. 10d illustrates a cross-section of a device 800′ comprising a disc 814′ having a plurality of peripheral apertures 819 in addition to a central aperture 816′.

The device 800 further includes an actuator 820 that couples the disc 814 to a driver 822, which provides energy to the actuator 820 and enables the actuator 820 to pivot the disc 814 through several degrees of activation (i.e., degrees of occlusion of the lumen 806). The actuator 820 and the driver 822 are positioned on one side of the flow restrictor 805. In the pictured embodiment, the actuator 820 and the driver 822 are positioned closer to the proximal end 810 than the distal end 812. In other embodiments, the actuator 820 and the driver 822 may be positioned on an opposite side of the flow restrictor 805, with the actuator 820 and the driver 822 positioned closer to the distal end 812 than the proximal end 810. As shown in FIG. 10c, in alternate embodiments, the device 800 may include a plurality of actuators and drivers positioned on both sides of the flow restrictor 805. In FIG. 10a, the actuator 820 extends along a longitudinal axis LA of the actuator 820 from the driver 822 to a position 824 located along an axis VA on a proximal face of 826 of the disc 814. In some embodiments, the axis LA also corresponds to the longitudinal axis of the device 800. The axis VA is substantially perpendicular to the axis LA.

The actuator 820 is shaped and configured as a linear actuator that shifts along the axis LA to transition the disc 814 through various degrees of activation. In the pictured embodiment, the actuator 820 is shaped and configured as an elongate rod that extends from the driver 822 to the disc 814. The actuator 820 may be any of a variety of linear actuators capable of applying a mechanical force to the disc 814 to tilt the disc 814 around the axis HA, including, but not limited to, a rod, a coil, a spring, and/or a lever. The actuator 820 may be formed of, by way of non-limiting example, a metallic material such as titanium or stainless steel, an elastomeric material, a polymeric material, a rubber material, a composite material, a shape memory material, a dielectric elastomer, a magnetic material, an electrostatic acrylic elastomer, or any other suitable flexible material to facilitate transitioning of the disc 814 between the active and inactive conditions. For example, in the pictured embodiment, the actuator 820 is formed of the shape-memory alloy Nitinol, which exhibits superelastic characteristics that facilitate applying mechanical force to the disc 814 to pivot it through various degrees of activation.

The disc 814 pivots within the device 800 about the axis HA in response to a mechanically induced force that is provided via selective actuation of the actuator 820 by the driver 800. Depending upon the signals and power received from other components of the device 800 (e.g., a microprocessor and/or power supply), the driver 822 influences the actuator 820 to appropriately tilt the disc 814 within the lumen 806 about an axis HA, which is substantially perpendicular to the axis VA. In FIG. 10a, the flow restrictor 805 is shown in an active condition, with a planar surface of the disc 814 being substantially planar to the axis VA. When the flow restrictor 805 is in an active condition, blood flow through the device 800 is partially blocked by the disc 814, and blood flow volume and flow rate through the device 800 is reduced, thereby creating a back pressure in the intravascular area 380 that activates the baroreceptors encircling the area 380.

When the driver 822 is signaled to shift the flow restrictor 805 into a less active condition, as shown in FIG. 10b, the driver 822 causes the actuator 820 to lengthen, thereby causing the position 824 of the disc 814 to tilt about the axis HA away from the driver 822 and the proximal end 810. As the flow restrictor tilts into a less active condition (i.e., as the disc 814 tilts away from the axis VA toward the axis HA), the amount of intraluminal occlusion decreases to allow blood to flow at an increased volume and rate through the device 800. This decrease in intraluminal occlusion relieves the back pressure in the area 380, thereby decreasing the activity of baroreceptor signaling in the area 380.

When the driver 822 is signaled to shift the flow restrictor 805 into an inactive condition, as shown in FIG. 10c, the drivers 822 cause the actuators 820 to lengthen sufficiently to cause the disc 814 to tilt until a planar surface (e.g., the proximal face 826) of the disc 814 is substantially aligned with and planar to the axis HA. When the flow restrictor 805 tilts into an inactive condition (i.e., as the disc 814 tilts away from the axis VA toward the axis HA), intraluminal occlusion is significantly minimized. Thus, when the flow restrictor 805 is in an inactive condition, blood flows through the lumen 806 of the device 800 with minimal disruption in blood volume and flow rate.

Conversely, when the driver 822 is signaled to shift the flow restrictor 805 into a more active condition, as shown in FIG. 10a, the driver 822 causes the actuator 820 to shorten, thereby causing the position 824 of the disc 814 to tilt about the axis HA away from the distal end 812 and toward the driver 822 and the proximal end 810. As the flow restrictor tilts into a more active condition (i.e., as the disc 814 tilts away from the axis HA toward the axis VA), the amount of intraluminal occlusion increases and blood flows at a decreased volume and rate through the device 800.

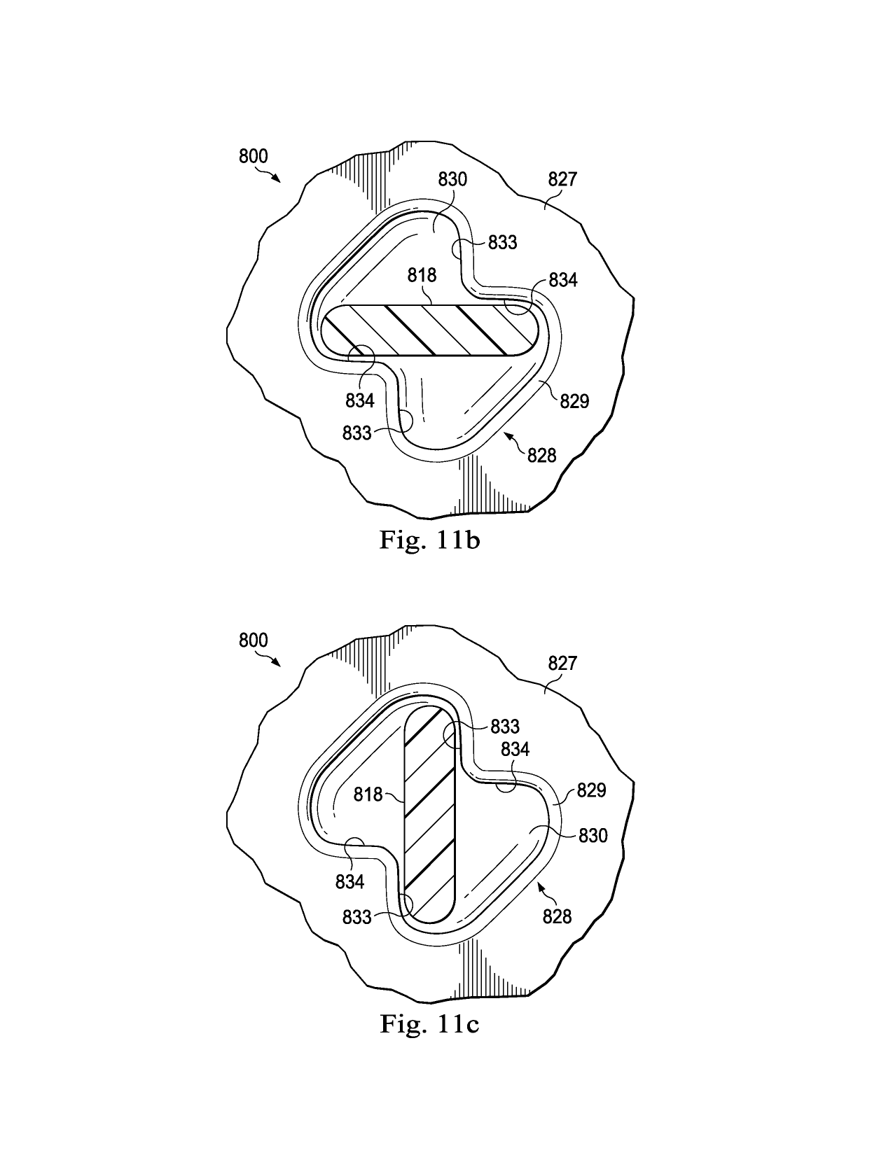

As mentioned above with reference to FIG. 10a, the side tabs 818 pivotably anchor the disc 814 within the support body 807 such that the disc 814 may pivot from an active position (as shown in FIG. 10a) to a less active (as shown in FIG. 10b) or inactive position (as shown in FIG. 10c). FIGS. 11a-11c illustrate one possible embodiment of the pivoting relationship between the disc 814, the side tabs 818, and the support body 807. As indicated in FIG. 11a, the support body 807 forms a hollow, generally cylindrical tube that houses the disc 814. The support body 807 may include thickened portions 827, which contain a pair of opposed recesses 828 for receiving the side tabs 818. Each recess 828 is a mirror image of the other. Each recess 828 is positioned along the axis HA within the luminal surface of one of the portions 827.

The contour and placement of the recesses 828 is selected to limit the range of movement of the side tabs 818 and the disc 814 between an active position (as illustrated in FIG. 11b) and an inactive position (as illustrated in FIG. 11c). Preferably, the recesses 828 have a sloped or curved circumferential edge 829 to facilitate the movement of blood through the recess and prevent stagnation of blood flow within the recess. Preferably, the recesses 828 also provide a curved or arcuate inner surfaces 830 for contact with the side tabs 818. Preferably, the side tabs 818 include correspondingly curved or arcuate outer surfaces 831 for contact with the inner surfaces 830. Preferably, the disc 814 includes curved or arcuate outer surfaces 832 for contact with the inner surfaces 830. By providing curved and arcuate edges on the recess edges 829, the side tabs 818, and the disc 814, blood flowing past the flow restrictor 805 may be less likely to experience flow disturbance, stagnation, or high shear stress (and platelet activation) along the edges of the flow restrictor 805 and the recesses 828. Thus, the risk of platelet aggregation and thrombus formation around the flow restrictor 805 may be reduced.

FIG. 11b illustrates the side tab 818 positioned within a recess 828 such that the disc 814 (and thus the flow restrictor 805) is in an active condition, reducing blood flow and flow rate through the device 800 to activate baroreceptors proximal to the device 800. Each recess 828 is shaped and configured to provide active stops 833 and inactive stops 834. The active stops 833 cooperate with the actuator 829 to prevent the side tab 818 (and thus the disc 814) from pivoting past a fully active position and/or spinning from the mechanical force of blood travelling through the device 800.

FIG. 11c illustrates the side tab 818 positioned within a recess 828 such that the disc 814 (and thus the flow restrictor 805) is in an inactive condition, allowing (and perhaps minimally reducing) blood flow and flow rate through the device 800 and relieving any intraluminal back pressure proximal to the device 800. The inactive stops 834 cooperate with the actuator 829 to prevent the side tab 818 (and thus the disc 814) from pivoting past a fully inactive position and/or spinning from the mechanical force of blood travelling through the device 800.

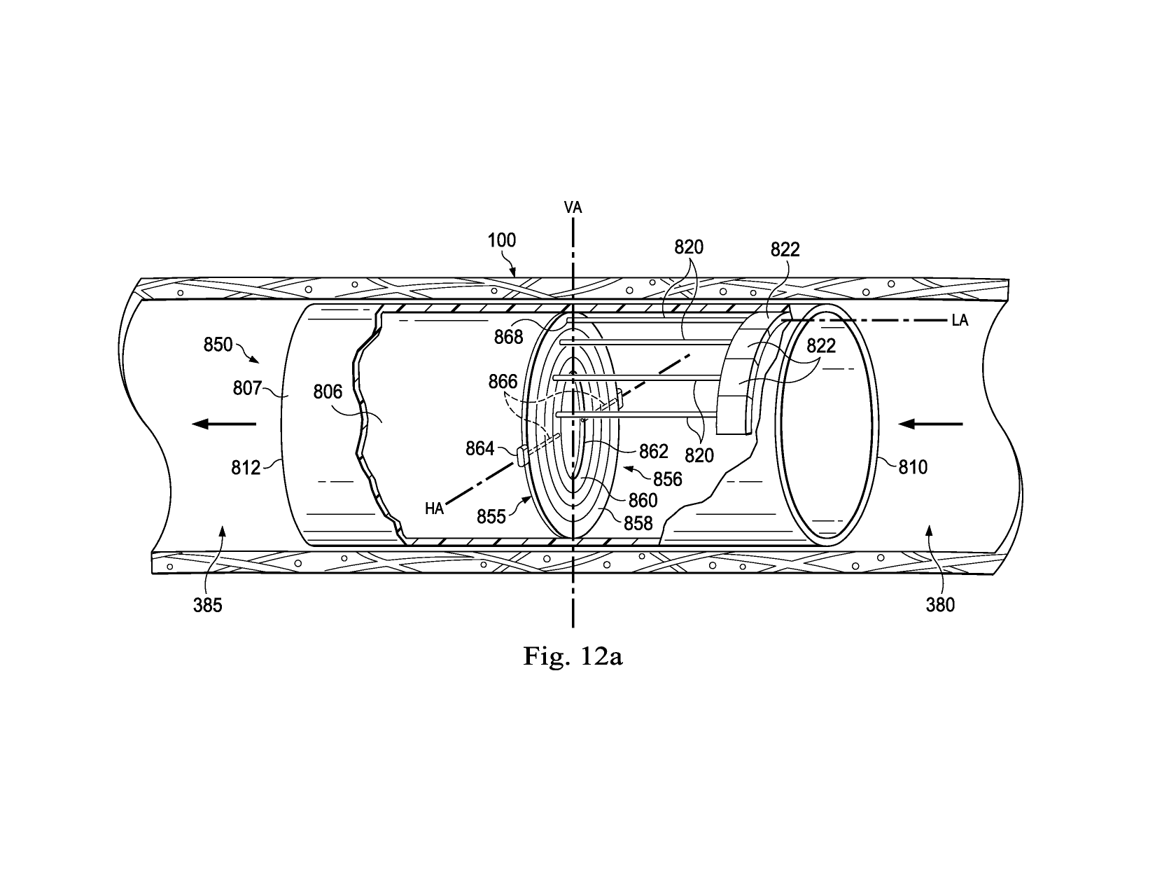

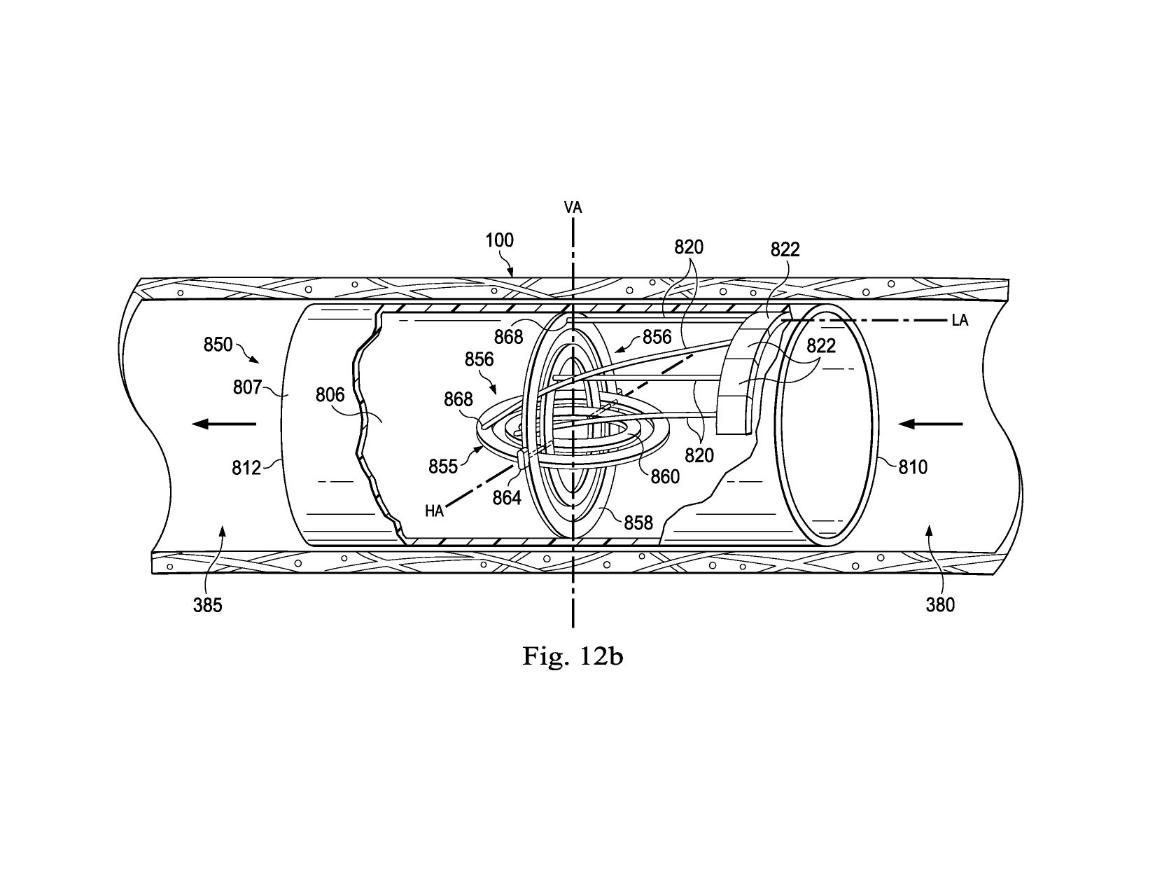

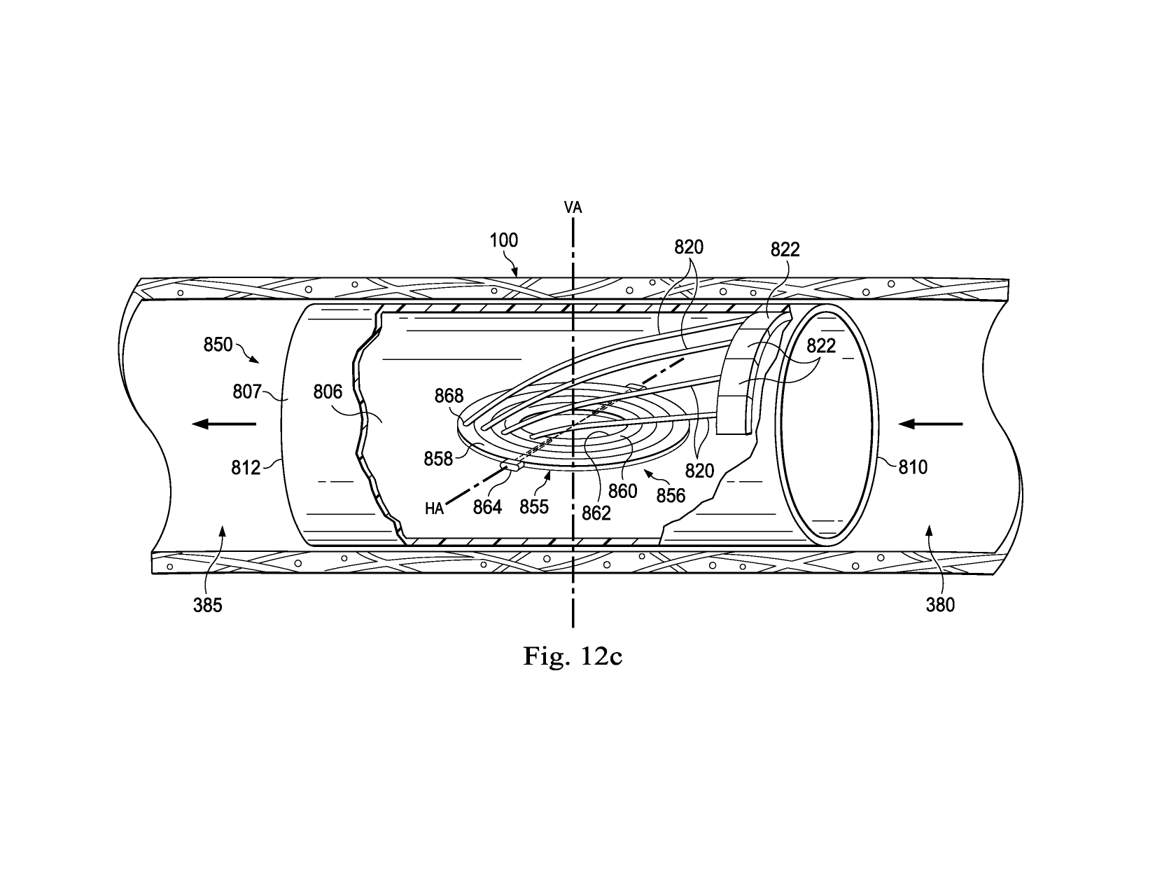

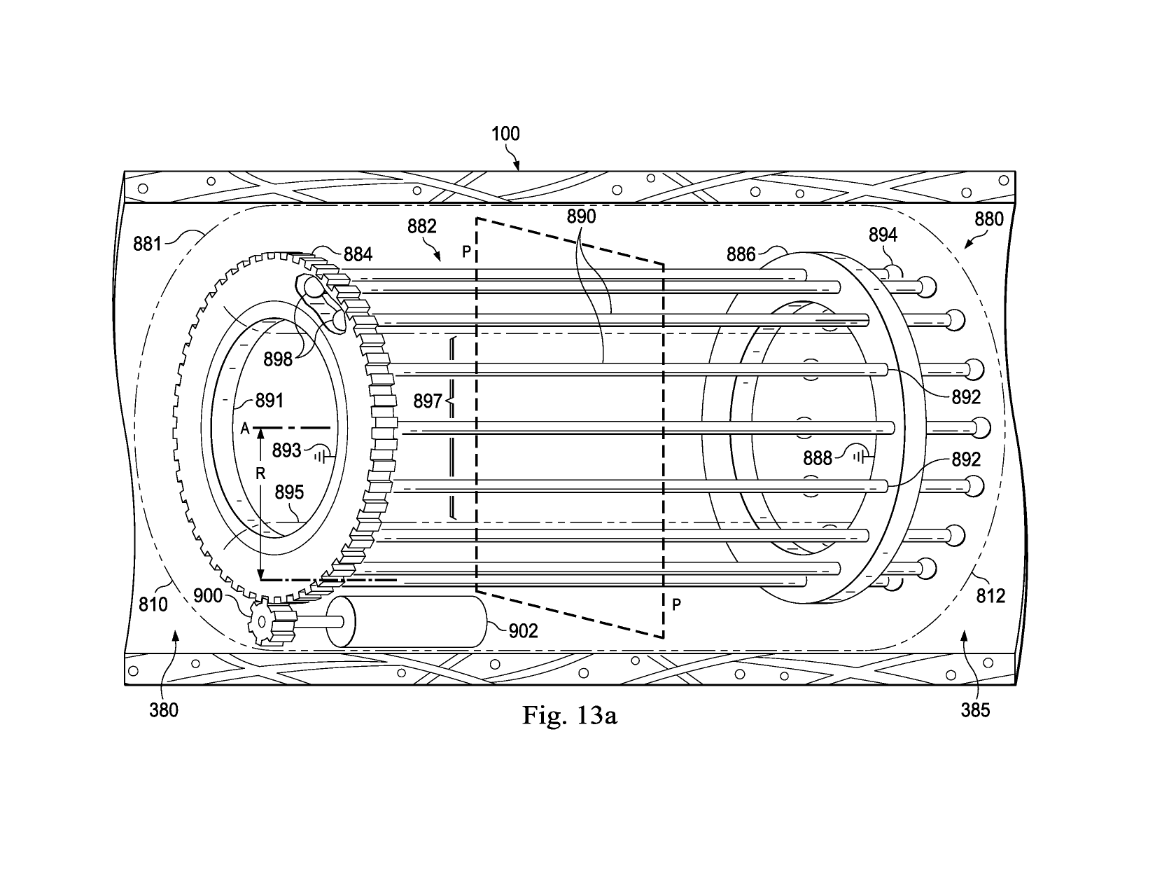

FIGS. 12a-12c illustrate an intravascular flow-modifying device 850 positioned within the vessel 100. The device 850 is shaped and configured substantially identical to the intravascular flow-modifying device 800 except for the differences noted herein. The device 850 includes a flow restrictor 855 positioned centrally within a lumen 806 and between the proximal end 810 and the distal end 812 of the elongate, hollow, cylindrical support body 807. The device 850 also includes a plurality of drivers 822 and actuators 820 coupled to the flow restrictor 855. The device 850 is shaped and configured for intravascular placement in a vessel such that the proximal end 810 is positioned upstream to the distal end 812, and blood flows from the intravascular area 380 proximal to the device 850, through proximal end 810, through the flow restrictor 855, and out the distal end 812 into the intravascular area 385 distal to the device 850.

The flow restrictor 855 includes a plurality of pivotable, concentric, circular rings 856 that gradually decrease in diameter from the outside ring 858 to the center ring 860. The center ring 860 includes a central aperture 862, and the outside ring 858 includes side tabs 864. The side tabs 864 are substantially identical to the side tabs 818 except for the differences noted herein. In the pictured embodiment, the flow restrictor 855 includes two rods 866, each of which extends from a side tab 864 through the plurality of concentric rings 856 to the central aperture 862. In some embodiments, the flow restrictor 855 may include only one rod that extends from one side tab 864, through the concentric rings 856 and the central aperture 862, to the other side tab 864. The side tabs 864 and the rods 866 pivotably anchor the plurality of concentric rings 856 within the support body 807 such that the concentric rings 856 may individually pivot about the rods 866 from an active position (as shown in FIG. 12a) to a less active (as shown in FIG. 12b) or inactive position (as shown in FIG. 12c).

The actuators 820 are shaped and configured as linear actuators that shift in a plane substantially parallel to an axis LA of each actuator 820 to transition the flow restrictor 855 through various degrees of activation. Each individual actuator 820 is coupled to a corresponding concentric ring 856 and a corresponding driver 822. Though the device 850 is shown including each individual driver 822 coupled to an individual actuator 820-ring 856 pair, other embodiments may include any number and combination of actuators, drivers, and rings. In FIG. 12a, each actuator 820 extends along the longitudinal axis LA from the corresponding driver 822 to a position 868 located on a proximal face of 870 of a concentric ring 856. Each individual concentric ring 856 may pivot within the device 850 about the axis HA in response to a mechanically induced force that is provided via selective actuation of the corresponding actuator 820 by the corresponding driver 800. Depending upon the signals and power received from other components of the device 850 (e.g., a microprocessor and/or power supply), various drivers 822 influence the corresponding actuators 820 to appropriately tilt particular rings 856 within the lumen 806 about the axis HA and/or the rods 866.

In FIG. 12a, the flow restrictor 855 is shown in an active condition, with the planar surfaces of the all the concentric rings 856 being substantially planar to the axis VA. When the flow restrictor 855 is in an active condition, blood flow through the device 800 is partially blocked by the plurality of concentric rings 856, and blood flow volume and flow rate through the device 850 is reduced, thereby creating a back pressure in the intravascular area 380 that activates the baroreceptors encircling the area 380. When the drivers 822 are signaled to shift the flow restrictor 855 into an active condition, the drivers 822 cause the corresponding actuators 820 to shorten, thereby causing the position 868 of the corresponding ring 856 to tilt about the axis HA away from the distal end 812 and toward the proximal end 810. As the flow restrictor 855 tilts into a more active condition, the amount of intraluminal occlusion increases and blood flows at a decreased volume and rate through the device 850.

When some drivers 822 are signaled to shift the flow restrictor 855 into a less active condition, as shown in FIG. 12b, the appropriate drivers 822 cause the corresponding actuators 820 to lengthen, thereby causing the positions 868 of the corresponding rings 856 to tilt about the axis HA away from the proximal end 810. As the flow restrictor tilts into a less active condition, the amount of intraluminal occlusion decreases to allow blood to flow at an increased volume and rate through the device 850. This decrease in intraluminal occlusion relieves the back pressure in the area 380, thereby decreasing the activity of baroreceptor signaling in the area 380.

When some drivers 822 are signaled to shift the flow restrictor 855 into an inactive condition, as shown in FIG. 12c, the appropriate drivers 822 cause the corresponding actuators 820 to lengthen sufficiently to cause the corresponding rings 856 to tilt until a planar surface (e.g., the proximal face 870) of the ring 856 is substantially aligned with and planar to the axis HA. When the flow restrictor 855 tilts into an inactive condition, intraluminal occlusion is significantly minimized. Thus, when the flow restrictor 855 is in an inactive condition, blood flows through the lumen 806 of the device 850 with minimal disruption in blood volume and flow rate.

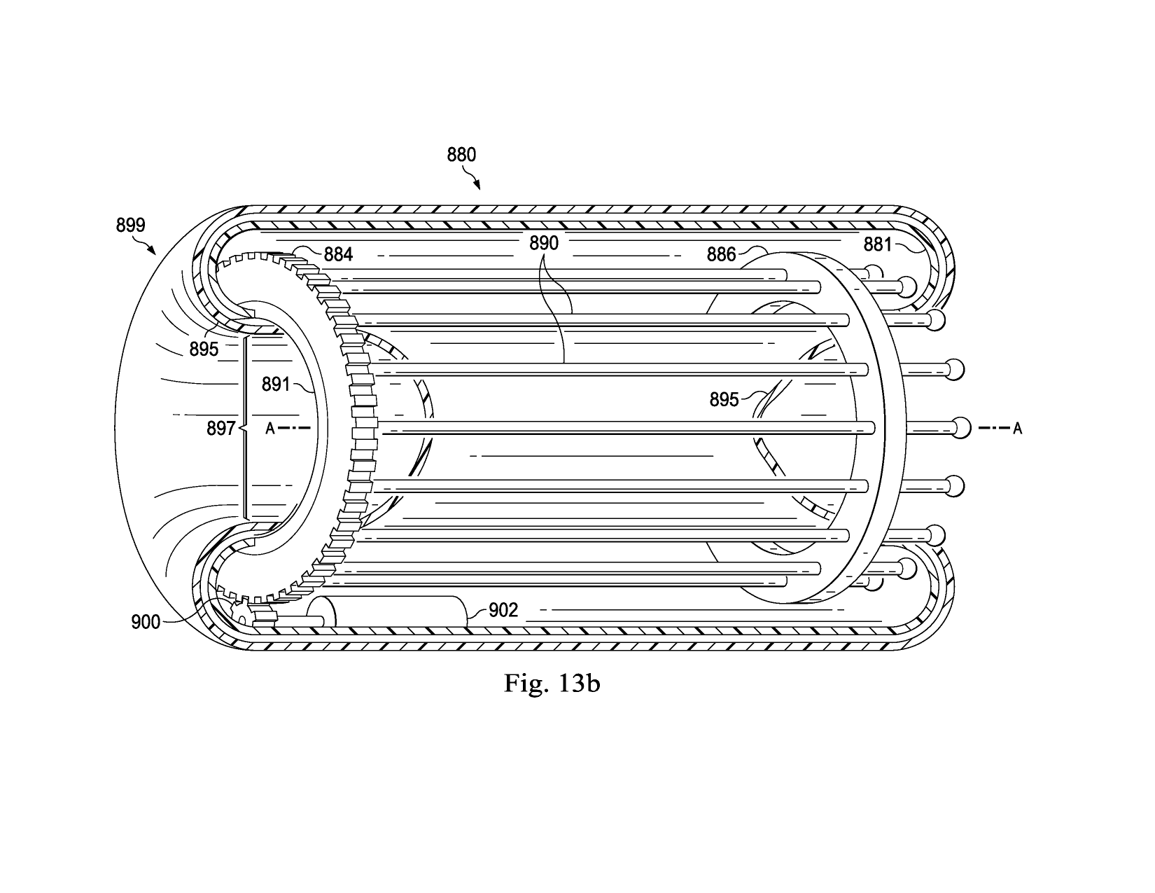

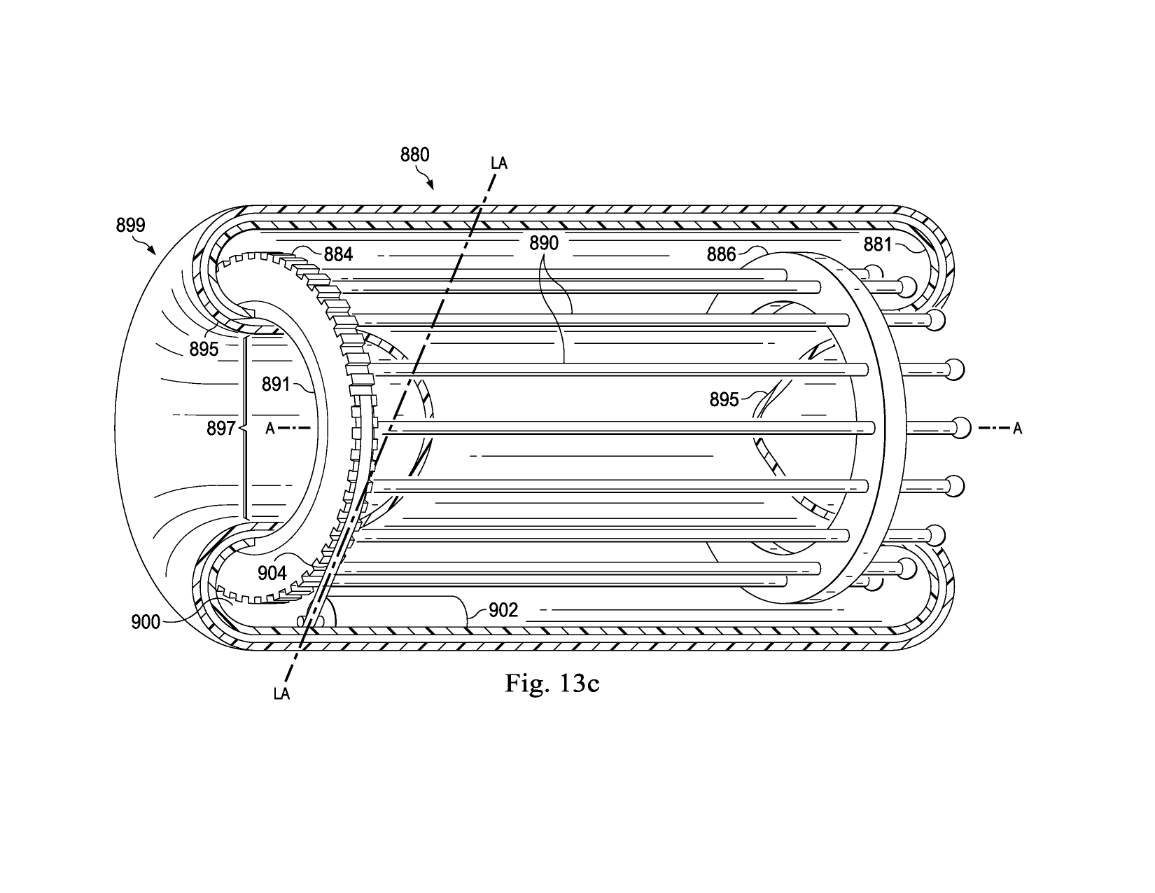

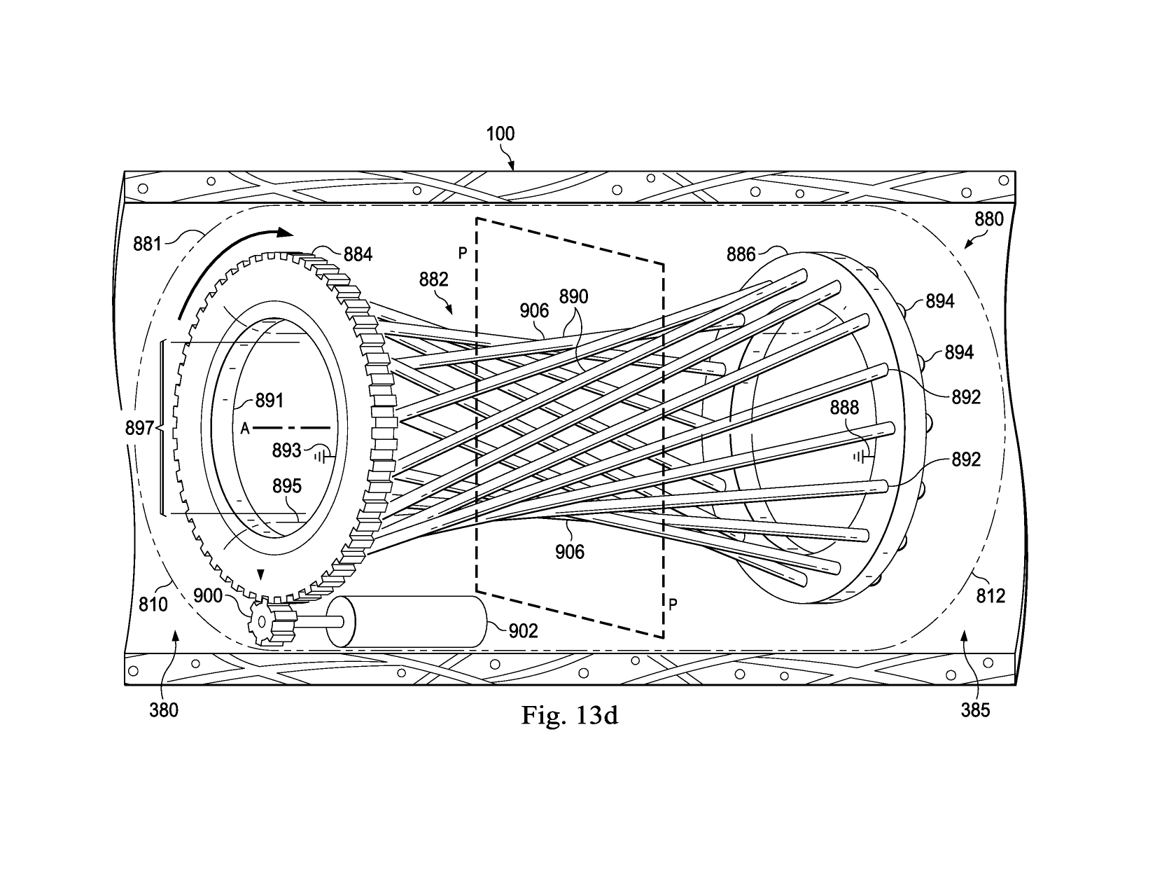

FIGS. 13a-13b illustrate an intravascular flow-modifying device 880 positioned within the vessel 100. The device 880 includes a flow restrictor 882 housed within the elongate, hollow, cylindrical support body 881. The device 880 includes a proximal end 810 and a distal end 812. The device 880 is shaped and configured for intravascular placement in a vessel such that the proximal end 810 is positioned upstream to the distal end 812, and blood flows from the intravascular area 380 proximal to the device 880, through proximal end 810, through the flow restrictor 882, and out the distal end 812 into the intravascular area 385 distal to the device 880.

The flow restrictor 882 includes a proximal ring 884, which is shaped and configured to rotate within the support body 881, a distal ring 886, which is shaped and configured to be stationary within the support body 881 (as indicated by the dashed lines 888), a plurality of rods 890, a bearing ring 891, which is configured to be stationary within the proximal ring 884 (as indicated by the dashed lines 893), and an inner sheath 895, which defines an inner lumen 897 of the device 880. The distal ring 886 anchors the flow restrictor 882 within the support body 881 such that the proximal ring 884 may rotate to transition the flow restrictor 882 from an inactive position (as shown in FIG. 13a) to a more active position (as shown in FIG. 13d). The plurality of rods 890 extend from the proximal ring 884 through aligned openings 892 in the distal ring 886 and cooperate with the proximal ring 884 and the distal ring 886 to selectively restrict blood flow through the device 880.

While the flow restrictor 882 is in an inactive condition, as shown in FIG. 13a, the rods 890 are positioned between the rings 884, 886 such that they pass in directions parallel to and spaced a given distance R from a longitudinal axis A-A of the flow restrictor 882 extending through the rings 884, 886. The rods 890 extend through the openings 892 in the distal ring 886 and terminate in rounded or curved distal ends 894 that lack sharp angles so as to minimize the potential for thrombogenesis and/or turbulent flow within the vessel 100. The proximal ends 896 (not shown) of the rods 890 are coupled to the proximal ring 884 by multi-axial joints 898, which permit the rods 890 to twist and/or tilt with respect to the axis A-A and a plane P of the rings 884, 886 (that is substantially perpendicular to the axis A-A). The rods 890 may be made of any of a variety of semi-rigid or rigid biocompatible materials, including, by way of non-limiting example, stainless steel, titanium, aluminum, polymeric composites, and/or plastic. The joints 898 may be any one of a variety of joint types, including, by way of non-limiting example, ball-and-socket joints and/or multi-axial screw joints.

The bearing ring 891 is positioned within the proximal ring 884 and supports the ring 884 for rotation in the plane P. The bearing ring 891 is shaped and configured to be stationary as the flow restrictor transitions from inactive to active (and visa-versa) conditions (as indicated by the dashed lines 902).

The inner sheath 895 extends from the bearing ring 891 to the distal ring 886 and separates the blood flowing through the device 880 from a length of the rods 890 positioned between the rings 884, 886. The inner sheath 895 is shaped and configured as a flexible, hollow, cylindrical tube that defines the lumen 897 of the device 880. The inner sheath 895 permits blood flow through the device 880 even when the flow restrictor 882 is in an active condition (as shown in FIG. 13d). In some embodiments, as illustrated in FIGS. 13b and 13c, the inner sheath 895 may comprise a continuous extension of the support member 881, wherein the inner sheath 895 and the support member 881 form a hollow, generally toroidal structure 899 encasing the flow restrictor 882 and separating the flow restrictor 882 from the bloodstream.

As illustrated in more detail in FIGS. 13b and 13c, the device 880 further includes an actuator 900 that couples the proximal ring 884 to a driver 902, which provides energy to the actuator 900 and enables the actuator 900 to rotate the proximal ring 884 through several degrees of activation (i.e., degrees of occlusion of the vessel 100). The actuator 900 and the driver 902 are substantially identical to the aforementioned actuator 820 and driver 822, respectively, unless otherwise disclosed herein. In the pictured embodiment, the actuator 900 and the driver 902 are positioned adjacent to the proximal ring 884 and within the toroidal structure 899. In other embodiments, the actuator 900 and the driver 902 may be elsewhere within the device 880, such as, by way of non-limiting example, within the lumen 897 against the inner sheath 895. In alternate embodiments, the device 880 may include a plurality of actuators and corresponding drivers.

The proximal disk 884 rotates within the toriodal structure 889 about the axis A-A in response to a mechanically induced force that is provided via selective actuation of the actuator 900 by the driver 902. Depending upon the signals and power received from other components of the device 880 (e.g., a microprocessor and/or power supply), the driver 902 influences the actuator 900 to appropriately rotate the proximal disk 884 to restrict blood flow through the lumen 897 of the device 880. As described in further detail below with respect to FIG. 13d, rotation of the proximal ring 884 from an inactive position to an active position causes restriction and occlusion of the lumen 897 of the device 880.

The actuator 900 may be any of a variety of actuators capable of applying a mechanical force to the proximal ring 884 to rotate the ring 884 around the axis A-A, including, but not limited to, a gear, a rod, a coil, a spring, and/or a lever. The actuator 900 may be formed of, by way of non-limiting example, a metallic material such as titanium or stainless steel, an elastomeric material, a polymeric material, a rubber material, a composite material, a shape memory material, a dielectric elastomer, a magnetic material, an electrostatic acrylic elastomer, or any other suitable flexible material to facilitate transitioning of the flow restrictor 882 between the active and inactive conditions.

For example, in the embodiment pictured in FIG. 13b, the actuator 900 is shaped as a circular pinion gear configured to meshingly engage with the proximal ring 884. In the pictured embodiment, the actuator 900 is preferably formed of a rigid or semi-rigid metal, polymer, or composite material, such as, by way of non-limiting example, titanium and/or stainless steel. When the driver 902 powers the actuator 900 to rotate in a first direction, the rotation of the actuator 900 impels the rotation of the proximal ring 884 in a second direction that is opposite to the first direction.

In the embodiment pictured in FIG. 13c, the actuator 900 is shaped as an elongate rod or cable configured to fixedly attach to a position 904 on the proximal ring 884. The actuator 900 is shaped and configured to extend along a longitudinal axis LA of the actuator 900 from the driver 902 to the position 904 on the ring 884. In the pictured embodiment, the actuator 900 may be formed of a self-expanding biocompatible material, such as Nitinol, a resilient polymer, a dielectric elastomer, an acrylic elastomer, or an elastically compressed spring temper biocompatible material. Other materials having shape memory characteristics, such as particular metal alloys, may also be used. When the driver 902 powers the actuator 900 to shift the proximal ring 884 into a more active position, the actuator 900 shortens along the axis LA, thereby shifting the position 904 toward to driver 902 and rotating the proximal ring 884 into a more active position.

FIG. 13d illustrates the intravascular flow-modifying device 880 in an active condition, wherein the proximal ring 884 is rotated into an active position, thereby decreasing the cross-sectional areas and diameters along the length of the lumen 897 and restricting blood flow through the device 880. FIG. 13d shows the effect of rotating the proximal ring 884 through a given angle as indicated by the curved arrow. Essentially, this rotation of the ring 884 twists the rods 890 and retracts them through the openings 892 in the distal ring 886. As the rods 890 retract through the openings 892, the ends 894 prevent the rods 890 from completely withdrawing from the ring 886. As a consequence of the rods 890 twisting, the given radial distance R (described in FIG. 13a) is decreased. Specifically, centers of the rods 890 move radially inward to reduce the passage area through the lumen 897 of the device 882. When the flow restrictor 882 is in an active condition, blood flow through the device 880 is delayed or partially blocked by the reduced passage size of the lumen 897, and blood flow volume and flow rate through the device 880 is reduced, thereby creating a back pressure in the intravascular area 380 that activates the baroreceptors encircling the area 380.

When the proximal ring 884 is returned to its original, inactive position (i.e., rotated back through the same given angle) as shown in FIG. 13a, then the rods 890 will expand outwardly to provide a maximum-sized passage through the lumen 897 of the device 880. For example, with reference to FIGS. 13c and 13a, when the driver 902 is signaled to shift the flow restrictor 882 into a less active condition, the proximal ring 884 rotates about the axis A-A away from the driver 902. As the flow restrictor 882 twists into a less active condition, the amount of intraluminal occlusion decreases to allow blood to flow at an increased volume and rate through the lumen 897 of the device 880. This decrease in intraluminal occlusion relieves the back pressure in the area 380, thereby decreasing the activity of baroreceptor signaling in the area 380. When the flow restrictor 882 is in an inactive condition, as shown in FIG. 13a, blood flows through the lumen 897 of the device 880 with minimal disruption in blood volume and flow rate.

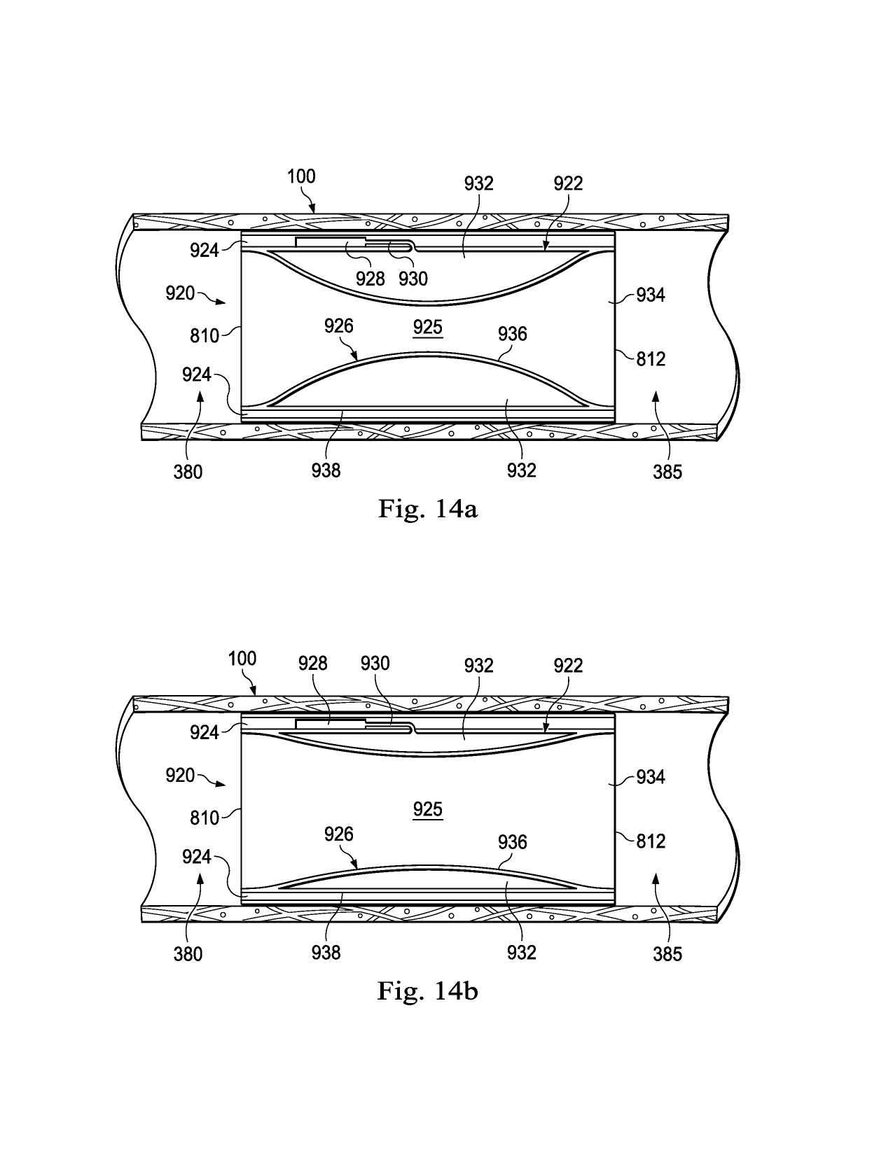

FIGS. 14a-14b illustrate an intravascular flow-modifying device 920 positioned within the vessel 100. FIG. 14a illustrates the device 920 in an active condition and FIG. 14b illustrates the device 920 in an inactive or less active condition. The device 920 includes a flow restrictor 922 housed within an elongate, hollow, cylindrical support body 924. The device 920 includes a lumen 925 that extends from a proximal end 810 to a distal end 812. The device 920 is shaped and configured for intravascular placement in a vessel such that the proximal end 810 is positioned upstream to the distal end 812, and blood flows from the intravascular area 380 proximal to the device 920, through the proximal end 810, through the flow restrictor 922, and out the distal end 812 into the intravascular area 385 distal to the device 920.

The flow restrictor 922 includes an expandable balloon 926 that is in fluid communication with a driver 928 by means of a hollow flow line 930. The driver 928 is shaped and configured as a pump to deliver a fluid or a gas through the flow line 930 into a hollow chamber 932 housed within the balloon 926. The driver pump 928 is configured to communicate with the communication module, microprocessor, and power supply of the device 920 in substantially an identical manner as the respective components of the device 300. Thus, in response to the appropriate command signals, the driver pump 928 may deliver an inflation medium, whether a fluid or a gas, through the flow line 930 into the chamber 932 to inflate the balloon 926 and transition the flow restrictor 922 (and the device 920) from an inactive condition (as shown in FIG. 14b) to a more active condition (as shown in FIG. 14a), and to deflate the balloon 926 and transition the flow restrictor 922 back to a less active or inactive condition (as shown in FIG. 14b).

In the pictured embodiment, the driver pump 928 and the fluid line 930 are embedded within the support body 924. In other embodiments, the driver 928 and the fluid line 930 may be positioned elsewhere within the device 920, such as, by way of non-limiting example, within the lumen 925 or within the expandable balloon 926. In alternate embodiments, the device 920 may include a plurality of actuators and corresponding driver pumps. In the pictured embodiment, the driver pump 928 includes a reservoir (not shown) containing the inflation medium. In other embodiments, the driver pump may be coupled to a separate reservoir of inflation medium positioned either within the device 920 or remote from the device 920.

In FIGS. 14a and 14b, the expandable balloon 926 is shaped and configured to have a generally annular and toroidal geometry including a central passageway 934. In the pictured embodiment, the central passageway 934 defines the lumen 925 of the device 920. The balloon 926 may include an interior surface 936 and a generally cylindrical exterior surface 938, which is circumferentially coupled to an entire inner circumference of the support body 924. In other embodiments, the balloon 926 may be shaped and configured to have a semi-spherical or semi-elliptical shape that resides on a portion of the support body 924 instead of the entire inner circumference of the support body 924. In such embodiments, multiple balloons may be utilized to provide greater degrees of occlusion of the lumen 925 of the device 920. However shaped and configured, the expandable balloon is shaped and configured to lack sharp angles so as to minimize the potential for thrombogenesis and/or turbulent flow within the vessel 100.

Depending upon the signals and power received from other components of the device 920 (e.g., a microprocessor, communication module, and/or power supply), the driver pump 928 appropriately inflates or deflates the balloon 926 to restrict or allow, respectively, blood flow through the lumen 925 of the device 920. When the driver pump 928 supplies inflation media to the chamber 932 of the balloon 926, the balloon 926 circumferentially expands or inflates, thereby transitioning the flow restrictor 922 into an active condition by narrowing the lumen 925, as shown in FIG. 14a. Narrowing the lumen 925 decreases the cross-sectional areas and diameters along the length of the lumen 925 and decreases the blood flow volume and rate through the device 920, which creates a back pressure in the area 380 proximal to the device 920 and activates the baroreceptors in the vicinity of area 380. It is important to note that the chamber 932 and the balloon 926 are configured to expand only to the extent that the flow restrictor 922 permits blood flow through the central passageway 934 even when the flow restrictor 922 is in an active condition.

When the driver pump 928 withdraws the inflation medium from the chamber 932, the balloon 926 is returned to its original, inactive condition with the interior surface 936 drawn towards the exterior surface 938 as shown in FIG. 14b. As the flow restrictor 922 transitions into a less active condition, the amount of intraluminal occlusion decreases to allow blood to flow at an increased volume and rate through the lumen 925 of the device 920. This decrease in intraluminal occlusion relieves the back pressure in the area 380, thereby decreasing the activity of baroreceptor signaling in the area 380. When the flow restrictor 922 is in an inactive condition, as shown in FIG. 14b, blood flows through the lumen 925 of the device 920 with minimal disruption in blood volume and flow rate.

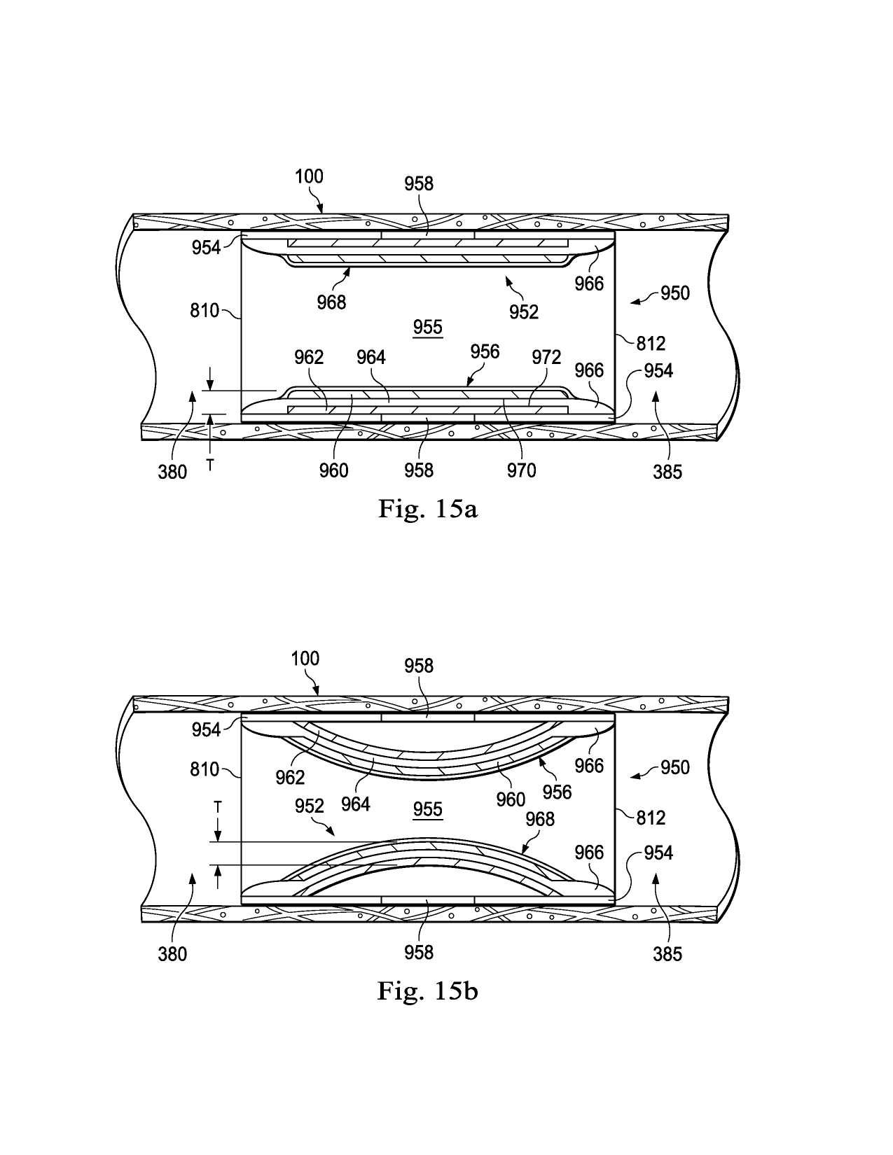

FIGS. 15a-15b illustrate an intravascular flow-modifying device 950 positioned within the vessel 100. FIG. 15a illustrates the device 950 in an inactive condition and FIG. 15b illustrates the device 920 in an active condition. The device 950 includes a flow restrictor 952 housed within an elongate, hollow, cylindrical support body 954. The device 950 includes a lumen 955 that extends from a proximal end 810 to a distal end 812 of the device 950. The device 950 is shaped and configured for intravascular placement in a vessel such that the proximal end 810 is positioned upstream to the distal end 812, and blood flows from the intravascular area 380 proximal to the device 950, through the proximal end 810, through the flow restrictor 952, and out the distal end 812 into the intravascular area 385 distal to the device 950.

The flow restrictor 952 includes at least one expandable structure 956 and at least one corresponding biasing member 958 that is configured to bias the expandable structure 956 away from the walls of the support body 954 toward the center of the lumen 955. The expandable structure 956 includes a first electrode 960, a polymeric film 962, and a second electrode 964. In the pictured embodiment, the device 950 includes at least two expandable structures 956 and at least two corresponding biasing members 958. In other embodiments, the flow restrictor may include any number of expandable structures and corresponding biasing members.

The biasing member 958 provides sufficient force to the expandable structure 956 to compel the expandable structure 956 to expand away from the support body 954 toward the lumen 955. In FIGS. 15a and 15b, the biasing member 958 is schematically depicted as a generic structure positioned adjacent the expandable structure 956 and within the support body 954. In various embodiments, the biasing member may be shaped and configured as any of a variety of biasing apparatuses, including, by way of non-limiting example, a spring, a stationary projection or series of projections extending from the support body 954 toward the lumen 955, and/or light pressure from a fluid/gas diaphragm (as described above with respect to FIGS. 14a and 14b). Other embodiments may lack a biasing member. For example, the expandable structure 95 may be shaped and configured to self-bias and expand in the appropriate direction, thus obviating the need for a separate biasing member 958.

The expandable structure 956 is shaped and configured as an electroactive polymer called a dielectric elastomer, which includes the first electrode 960 and the second electrode 962 sandwiched around the polymeric film 964. The polymeric film 964 extends beyond the electrodes 960, 962 to couple the expandable structure 956 to the support body 954 at the margins 966 of the expandable structure 956. The expandable structure includes an active area 968 that includes the electrodes 960, 962 and extends between the margins 966. The active area 968 deflects from the support body 954 when the flow restrictor 952 is in an active condition to restrict the lumen 955 of the device 950. The electrodes 960, 962 comprise compliant electrodes made of any of a variety of suitable materials, such as, by way of non-limiting example, carbon particles suspended in a soft polymer matrix. The electrodes 960, 962 are electrically coupled to the power supply (e.g., 615, not shown here for the sake of simplicity) and/or a driver (e.g., 605, not shown here for the sake of simplicity). In response to the appropriate command signals and/or power supply, the expandable structure 956 is activated (i.e., energized) to expand the active area and transition the flow restrictor 952 (and the device 950) from an inactive condition (as shown in FIG. 15a) to a more active condition (as shown in FIG. 15b), and deactivated to unexpand the active area and transition the flow restrictor 952 back to an inactive condition.

It is important to note that the expandable structure 956 may convert between electrical energy and mechanical energy bi-directionally. For example, the expandable structure 956 may comprise an electrical generator because the expandable structure is configured to produce a change in electric field in response to deflection of the expandable structure. Specifically, the change in electric field, along with changes in the polymer dimension in the direction of the field, produces a change in voltage, and hence a change in electrical energy. When deflection of the active area 968 toward the support body 954 causes the net area of the active area 968 to decrease and there is a charge on the electrodes 960, 962, the active area 968 acts as a generator by converting mechanical energy into electrical energy. Conversely, when the deflection away from the support body causes the net area of the active area 968 to increase and charge is on the electrodes, the active area 968 acts as an actuator by converting electrical energy to mechanical energy. The change in area in both cases corresponds to a reverse change in the thickness T of the active area 968, i.e., the thickness T contracts when the planar area expands (as shown in FIG. 15b), and the thickness expands when the planar area contracts (as shown in FIG. 15a). Thus, devices of the present disclosure may include both actuator/mechanical and generator modes, depending on how the expandable structure 956 is arranged and utilized.

In some embodiments, the device 950 may store or harness the energy generated by the cyclical movement of the expandable structure 956 to power various components of the device 950, including the expandable structure itself.

Electroactive polymers deflect when actuated by electrical energy. In the pictured embodiment, the polymeric film 964 may comprise an electroactive polymer that acts as an insulating dielectric between the two electrodes 960, 962 and may deflect upon application of a voltage difference between the two electrodes. The first electrode 960 and the second electrode 962 are attached to the film 964 on its first surface 970 and second surface 972, respectively, to provide a voltage difference across the active area 968. Depending upon the signals and power received from other components of the device 950 (e.g., a microprocessor, communication module, and/or power supply), the driver 605 and/or power supply 615 appropriately energizes or deenergizes the expandable structure 956 to restrict or allow, respectively, blood flow through the lumen 955 of the device 950.

When electrical energy is supplied to the electrodes 960, 962 of the expandable structure 956, the active area 968 deflects away from the support body 954 into the lumen 955, thereby transitioning the flow restrictor 952 into an active condition by narrowing the lumen 955, as shown in FIG. 15b. Energy supplied to the electrodes 960, 962 causes a change in the electric field, thereby activating the active area 968, which deflects away from the support body 954 to assume a convex shape extending into the lumen 955. As the film 964 deflects toward the lumen 955, the thickness T of the active area 968 decreases as the unlike electrical charges produced by electrodes 960, 962 attract each other and provide a compressive force between electrodes 960, 962 and an expansion force on the film 964 in planar directions toward the circumferential edges of the active area 968, causing the active area 968 to compress between electrodes 960, 962 and stretch in the planar directions. Narrowing the lumen 955 decreases the cross-sectional areas and diameters along the length of the lumen 955 and decreases the blood flow volume and rate through the device 950, which creates a back pressure in the area 380 proximal to the device 950 and activates the baroreceptors in the vicinity of area 380. It is important to note that the expandable structure 956 is configured to expand only to the extent that the flow restrictor 952 permits blood flow through the lumen 955 even when the flow restrictor 952 is in an active condition.

In general, the active area 968 continues to deflect until mechanical forces balance the electrostatic forces driving the deflection. The mechanical forces include, by way of non-limiting example, elastic restoring forces of the film 964 material, the compliance of the electrodes 960, 962, and/or any external resistance provided by a device and/or load coupled to the active area (e.g., biasing member 958). The deflection of the active area 968 as a result of the applied voltage may also depend on a number of other factors such as the dielectric constant of the film 964 and the dimensions of the film 964.

The electrodes 960, 962 are compliant and change shape with the film 964. The configuration of the film 964 and the electrodes 960, 962 provides for increasing active area 968 response with increasing deflection away from the support body 954. In some embodiments, the expandable structure 956 is incompressible, i.e., has a substantially constant volume under stress. In these embodiments, the active area 968 decreases in thickness as a result of the expansion in the planar directions. More specifically, as the active area 968 deflects into a more active condition as shown in FIG. 15b, compression of the film 964 brings the opposite charges of the electrodes 960, 962 closer together and the substantially simultaneous stretching of film 964 separates similar charges in each electrode. In one embodiment, one of the electrodes 960, 962 functions as a ground electrode.

As shown in FIG. 15a, when the driver 605 and/or power supply 615 withdraws electrical energy from the electrodes 960, 962, the active area 968 is returned to its original, flattened, inactive condition against the support body 954. More specifically, the removal of the voltage difference and the induced charge causes the active area 968 to flatten toward the support body 954 and the thickness T to increase. As the flow restrictor 952 transitions into a less active condition, the amount of intraluminal occlusion decreases to allow blood to flow at an increased volume and rate through the lumen 955 of the device 950. This decrease in intraluminal occlusion relieves the back pressure in the area 380, thereby decreasing the activity of baroreceptor signaling in the area 380. When the flow restrictor 952 is in an inactive condition, as shown in FIG. 15a, blood flows through the lumen 955 of the device 950 with minimal disruption in blood volume and flow rate.

Various exemplary materials suitable for use in the expandable structure 956 include, by way of non-limiting example, silicone elastomers, acrylic elastomers, polyurethanes, thermoplastic elastomers, copolymers comprising PVDF, pressure-sensitive adhesives, fluoroelastomers, polymers comprising silicone and acrylic moieties, and the like. Polymers comprising silicone and acrylic moieties may include copolymers comprising silicone and acrylic moieties, and polymer blends comprising a silicone elastomer and an acrylic elastomer, for example. Combinations of some of these materials may also be used in some embodiments of the present disclosure.

Although the discussion has focused primarily on one type of electroactive polymer commonly referred to as dielectric elastomers, expandable structures 956 of the present disclosure may also incorporate other conventional electroactive polymers. As the term is used herein, an electroactive polymer refers to a polymer that responds to electrical stimulation. Other common classes of electroactive polymer suitable for use with various embodiments of the present disclosure include, by way of non-limiting example, electrostrictive polymers, electronic electroactive polymers, and ionic electroactive polymers, and some copolymers. Electrostrictive polymers are characterized by the non-linear reaction of a electroactive polymers (relating strain to E2). Electronic electroactive polymers typically change shape or dimensions due to migration of electrons in response to electric field (usually dry). Ionic electroactive polymers are polymers that change shape or dimensions due to migration of ions in response to electric field (usually wet and contains electrolyte).

In some embodiments, multiple expandable structures 956 may be utilized to provide greater degrees of occlusion of the lumen 955 of the device 950. However shaped and configured, the expandable balloon is shaped and configured to lack sharp angles so as to minimize the potential for thrombogenesis and/or turbulent flow within the vessel 100.

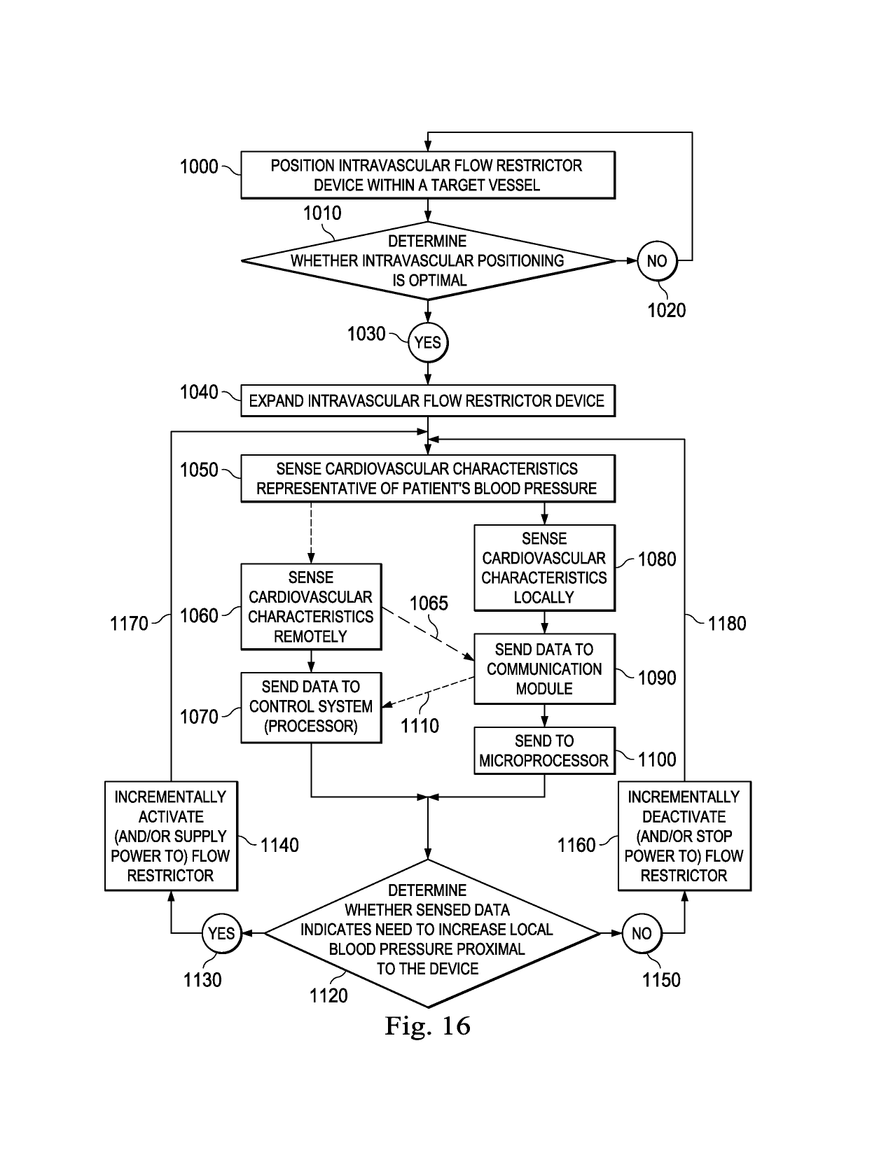

FIG. 16 provides a schematic flowchart illustrating methods of controlling blood pressure using an intravascular flow-modifying device of the present disclosure, e.g., device 300. All of the embodiments of intravascular flow-modifying devices disclosed herein are suitable for implantation, and are preferably implanted using a minimally invasive percutaneous and intravascular approach. The intravascular flow-modifying devices may be positioned anywhere within the venous or arterial vasculature where baroreceptors capable of modulating the baroreflex system are present. The intravascular flow-modifying devices will generally be implanted such that the device is positioned within a vessel immediately distal to a target area of the baroreceptors. For the purposes of illustration only, the methods disclosed by FIG. 16 will be discussed with respect to FIG. 4, which illustrates the intravascular flow-modifying device 300 positioned within the right renal vein 430.

In FIG. 16, step 1000 initiates the blood pressure control process with the user positioning the intravascular flow-modifying device 300 within the right renal vein 430. Prior to insertion of the device 300, a delivery apparatus, e.g., a guidewire, may be introduced into the arterial vasculature of a patient using standard percutaneous techniques. For example, once the guidewire is positioned within the target blood vessel, which is the right renal vein 430 in the illustrated embodiment of FIG. 4, the device 300 may be introduced in an unexpanded condition into the vasculature of a patient over the guidewire and advanced to the area of interest. In the alternative, the device 300 may be releasably coupled in an unexpanded condition to the delivery apparatus external to the patient and both the guidewire and the device 300 may be simultaneously introduced into the patient and advanced to the vessel of interest.

The device 300 is implanted within the renal vasculature such that the device 300, which is disposed in an unexpanded condition when introduced into the patient's vasculature, is positioned distal to the target baroreceptors of interest (e.g., baroreceptors 110 illustrated in FIG. 3). At step 1010, the user may determine whether the device 300 is optimally positioned within the vessel. The delivery apparatus may include IVUS or other imaging apparatuses thereon, thereby permitting the user to precisely position the device 300 within the blood vessel by using in vivo, real-time intravascular imaging. Additionally or alternatively, the user may utilize external imaging, such as, by way of non-limiting example, fluoroscopy, ultrasound, CT, or MRI, to aid in the guidance and positioning of the device 300 within the patient's vasculature.

At step 1020, if the device 300 is not optimally positioned within the vessel, the user may reposition the device 300 within the vessel at step 1000 and recheck the position at step 1010.

After step 1030, when the user determines that the device 300 is optimally positioned within the vessel, the user may expand the intravascular flow-modifying device 300 within the vessel immediately distal to the baroreceptors of interest at step 1040. Expansion of the stent-like support body 600 of the device 300 preferably anchors the device against the vessel walls by applying a biasing force against the vessel walls (e.g., vessel walls 120 illustrated in FIG. 3). In the neutral, unactivated and/or unpowered condition, the flow restrictor 360 of the device 300 assumes an inactive condition that does not significantly alter flow through the device 300.

With reference to FIGS. 5 and 16, at step 1050, the user and/or control system 505 may direct any of the sensors associated with the blood pressure control system 500 to sense and/or monitor a cardiovascular characteristic or parameter representative of the patient's blood pressure and/or indicative the need to modify the activity of the baroreflex system (e.g., baroreflex system 160 illustrated in FIG. 2).

At step 1060, the user and/or control system 505 may activate and/or use any of the remote sensors 515 of the system 500 and direct them to sense and/or monitor a cardiovascular characteristic or parameter representative of the patient's blood pressure and/or indicative the need to modify the activity of the baroreflex system 160. In some embodiments, the remote sensor 515 may comprise an external blood pressure cuff. In other embodiments, the remote sensor 515 may comprise an internal sensor positioned within the patient's body such that it is capable of sensing cardiovascular characteristic or parameter representative of the patient's blood pressure and/or indicative the need to modify the activity of the baroreflex system 160.

At step 1070, the sensor 515 may generate a data signal indicative of the sensed parameter data and send the data signal to the control system 505 (in particular, to the processor 320) for processing. Additionally or alternatively, at step 1065, the sensor 515 may send the data signal to the communication module 620 of the device 300 for internal, local processing by the microprocessor 610.

Additionally or alternatively, at step 1080, the user and/or control system 505 may activate any of the local sensors of the system 500, including the onboard sensors 370, 372 and any auxiliary sensors 625, and direct them to sense and/or monitor a cardiovascular characteristic or parameter representative of the patient's blood pressure and/or indicative the need to modify the activity of the baroreflex system 160. In some instances, the user and/or control system 505 may only activate any of the local sensors of the system 500 if deemed necessary after evaluating the data signal sent by the remote sensors 515.

At step 1090, the local sensors 370, 372, and/or 625 may generate a data signal indicative of the sensed parameter data and send the data signal to the communication module 620 of the device 300.

At step 1100, the communication module 620 may send the data signal to the on-board microprocessor 610 for local processing. Additionally or alternatively, at step 1110, the communication module 620 may send the data signal to the control system 505 (in particular, to the processor 320) for remote processing.

At step 1120, any of the remote or local processors of the system 500 (e.g., the processor 320 and the microprocessor 610) and/or the user determines whether the sensed data indicates a need to increase the local blood pressure proximal to the device 300 to activate the baroreceptors 110 proximal to the device.

If, at step 1130, the system 500 and/or the user determine that the sensed data indicates a need to increase the local blood pressure proximal to the device 300, then, at step 1140, the system 500 and/or the user incrementally activates and/or supplies power to the flow restrictor 360 of the device 300, thereby incrementally increasing the degree of occlusion of the lumen 630 of the device 300 and increasing the back pressure proximal to the device 300. For example, if the sensed data indicates a globally hypertensive situation or a locally hypotensive situation that is unsafe for tissue health, the system 500 and/or the user may activate the flow restrictor 360 at step 1140. Activating the flow restrictor 360 may induce a baroreceptor signal from the area proximal to the device 300 that is perceived by the brain to be excessive blood pressure, which induces the brain to alter the activities of the baroreflex system 160 to decrease blood pressure.

If, at step 1150, the system 500 and/or the user determine that the sensed data does not indicate a need to increase the local blood pressure proximal to the device 300, then, at step 1160, the system 500 and/or the user incrementally deactivates and/or stops or decreases power to the flow restrictor 360 of the device 300, thereby incrementally decreasing the degree of occlusion of the lumen 630 of the device 300 and decreasing the back pressure proximal to the device 300. For example, if the sensed data indicates a globally hypotensive or normotensive situation or a locally hypertensive situation that is unsafe for tissue health, the system 500 and/or the user may deactivate the flow restrictor 360 at step 1160. Deactivating the flow restrictor 360 may reduce the baroreceptor signals from the area proximal to the device 300. Reduced baroreceptor activity may be perceived by the brain to be normal or low blood pressure, which induces the brain to alter the activities of the baroreflex system 160 to either maintain or increase, respectively, blood pressure.

At steps 1170 and 1180, the cycle may continue according to power conservation algorithms determined by the system 500 and/or the desires of the user, with the system 500 and/or the user directing any of the sensors associated with the blood pressure control system 500 to sense and/or monitor a cardiovascular characteristic or parameter representative of the patient's blood pressure and/or indicative the need to modify the activity of the baroreflex system.

Persons of ordinary skill in the art will appreciate that the embodiments encompassed by the present disclosure are not limited to the particular exemplary embodiments described above. In that regard, although illustrative embodiments have been shown and described, a wide range of modification, change, and substitution is contemplated in the foregoing disclosure. For example, the thermal basket catheter may be utilized anywhere with a patient's vasculature, both arterial and venous, having an indication for thermal neuromodulation. It is understood that such variations may be made to the foregoing without departing from the scope of the present disclosure. Accordingly, it is appropriate that the appended claims be construed broadly and in a manner consistent with the present disclosure.

We claim:

1. A method of treating hypertension, comprising:
implanting a flow restricting device in the vasculature of a patient;
sensing blood pressure;
actuating the flow restricting device in response to the sensed blood pressure to modify the flow of blood through the flow restrictor; and
sensing the blood pressure after said actuating to determine the effect of the modification of the blood flow.

2. The method of claim 1, wherein the flow restricting device includes a sensor and said sensing includes activating the onboard sensor.

3. The method of claim 1, wherein the flow restricting device includes an electrical actuator and the step of actuating includes sending an electrical signal to the actuator.

4. The method of claim 2, wherein the flow restricting device includes a power supply, and the sensing step includes powering the sensor.

5. The method of claim 1, wherein the flow restricting device includes a power harvesting mechanism, the method further including harvesting power from the body and using the power for at least one of actuating or sensing.

6. A vascular flow regulation device, comprising:
an anchoring body configured for fixed engagement with an vascular wall;
a flow constriction element coupled to the anchoring body, the flow constriction element movable between a high flow position and a low flow position; and
an actuator coupled to the flow constriction element, the actuator configured to move the flow constriction element between the high flow position and the low flow position.

7. The device of claim 6, wherein the actuator is a electrically powered.

8. The device of claim 7, further including a power supply carried by the anchoring body.

9. A vascular flow regulation device, comprising:
an anchoring body configured for fixed engagement with an vascular wall;
a flow constriction element coupled to the anchoring body, the flow constriction element movable between a high flow position and a low flow position; and
a sensing element coupled to the anchoring body and configured to detect at least one biometric parameter.

10. The device of claim 9, wherein the sensing element generates a signal and further including an actuator joined to the flow constricting device for moving the flow constricting element between the high flow and low flow positions in response to the signal.

11. The device of claim 9, wherein the sensing element sense blood pressure.

12. The device of claim 6, wherein the actuator is configured to return to the high flow condition in the absence of power.
